(19) United States
(12) Patent Application Publication
(10) Pub. No.: US 2003/0059465 A1
(43) Pub. Date: Mar. 27, 2003

(54) Stabilized nanoparticle formulations of camptotheca derivatives

(76) Inventors: Evan C. Unger, Tucson, AZ (US); Marek J. Romanowski, Tucson, AZ (US); VaradaRajan Ramaswami, Tucson, AZ (US)

Correspondence Address: REED & ASSOCIATES, 800 MENLO AVENUE, SUITE 210, MENLO PARK, CA, 94025, US

(21) Appl. No.: 10/165,867

(22) Filed: Jun. 6, 2002

Related U.S. Application Data
(63) Continuation-in-part of application No. 09/703,484, filed on Oct. 31, 2000.
Application No. 09/703,484 is a continuation-in-part of application No. 09/478,124, filed on Jan. 5, 2000.
Application No. 09/478,124 is a continuation-in-part of application No. 09/075,477, filed on May 11, 1998.

(51) Int. Cl. A61K 31/4745; A61K 9/127; A61K 9/20; A61K 9/14

(52) U.S. Cl. 424/465; 424/486; 514/283

(57) ABSTRACT

Pharmaceutical formulations are provided that increase the systemic bioavailability of camptotheca derivatives; preferably, the camptothecin derivative is 7-ethyl-10-hydroxyl camptothecin, SN-38. The drug is complexed with a stabilizing agent, but is not covalently bound thereto. Anionic or neutral lipids and/or polymers are used as the stabilizing agent, and secondary stabilizing agents and/or other excipients may be incorporated into the formulation as well. Therapeutic methods are also provided, wherein a formulation of the invention is administered to a patient to treat a condition, disorder, or disease that is responsive to camptothecin derivatives. Generally, administration is oral or parenteral.

### CROSS-REFERENCE TO RELATED APPLICATIONS

This is a continuation-in-part of U.S. patent application Ser. No. 09/703,484, filed Oct. 31, 2000, which is a continuation-in-part of U.S. patent application Ser. No. 09/478,124, filed Jan. 5, 2000, which is a continuation-in-part of U.S. patent application Ser. No. 09/075,477, filed May 11, 1998, the disclosures of which are incorporated herein by reference.

### TECHNICAL FIELD

The present invention relates generally to pharmaceutical formulations, and more particularly to pharmaceutical formulations containing nanoparticles that are preferably amorphous or noncrystalline, stabilized by polymers and/or lipids for the delivery of camptothecin analogs, preferably SN-38. The invention has utility in the fields of pharmaceutical formulation, drug delivery, and medicine.

### BACKGROUND

Camptothecin is an antineoplastic drug that acts an inhibitor of DNA topoisomerase. Several natural and synthetic analogs of camptothecin have been identified and tested for antineoplastic efficacy. Camptothecin derivatives, as a class, are insoluble in aqueous solvents, unless modified with polar or ionic groups, as is done with CPT-11. Thus, one approach to the pharmaceutical formulation of camptothecin derivatives has been to covalently modify them with polar or charged moieties to increase their water solubility. Drawbacks to this approach include reductions in therapeutic potency and the expense involved in synthesizing analogs.

The formulation and administration of water-insoluble or sparingly water-soluble drugs, such as camptothecin and camptothecin analogs, are problematic in general because of the difficulty of achieving sufficient systemic bioavailability. Low aqueous solubility results not only in decreased bioavailability, but also in formulations that are insufficiently stable over extended storage periods. For the most part, research has focused on entrapment of the drug in vesicles or liposomes, and on the incorporation of surfactants into camptothecin formulations.

Representative liposomal drug delivery systems are described in U.S. Pat. Nos. 5,395,619, 5,340,588, and 5,154,930. Liposomes, as is well known in the art, are vesicles comprised of concentrically ordered lipid bilayers that encapsulate an aqueous phase. Liposomes form when phospholipids (amphipathic compounds having a polar (hydrophilic) head group covalently bound to a long-chain aliphatic (hydrophobic) tail) are exposed to water. That is, in an aqueous medium, phospholipids aggregate to form a structure in which the long-chain aliphatic tails are sequestered within the interior of a shell formed by the polar head groups. Unfortunately, use of liposomes for delivering many drugs has proven unsatisfactory, in part because liposome compositions are, as a general rule, rapidly cleared from the bloodstream. Finally, even if satisfactory liposomal formulations could be prepared, it might still be necessary to use some sort of physical release mechanism so that the vesicle releases the camptothecin analog in the body before the liver and spleen take up the agent.

Specifically regarding liposomal delivery of camptothecin, Burke (U.S. Pat. No. 5,552,156) describes liposomes that have molar ratios of lipid to drug in the range of 1,000:1 to 100,000:1, or that have lipid concentrations of approximately 0.29 M. This range of ratios does not optimize the amount of antineoplastic agent for therapeutic administration, and the concentration makes stable vesicle formation problematic. Secondarily, liposome clearance would not allow for extended periods of bioavailability, without engineering the liposomes to avoid the reticuloendothelial system. While the use of PEGylated liposomes partially resolves this dilemma, the amounts of lipids needed for administration may give rise to acute toxicity.

Micelles also have been used for drug delivery, as exemplified by the disclosure in U.S. Pat. No. 5,736,156 regarding camptothecin. Micelles are defined as spherical receptacles comprised of a single monolayer defining a closed compartment. Generally, amphipathic molecules such as surfactants and fatty acids spontaneously form micellar structures in polar solvents. In contrast to liposome bilayers, micelles are “sided” in that they project a hydrophilic, polar outer surface and a hydrophobic interior. Since they are monolayers, they are extremely limited in size, seldom exceeding 30 nanometers in diameter. This limited size reduces their effective encapsulation potential as drug carriers.

Among other notable drug delivery formulations, nanocrystals of drugs or carrier-stabilized drugs have been described in the art (for example, in U.S. Pat. No. 5,399,363 to Liversidge et al.). Liversidge et al. describe the production of nanoparticles of hydrophobic drugs, including natural camptothecin, using surfactants and grinding. They mention a number of surfactants, including poloxamers, and list lecithin as a stabilizing material, but provide no disclosure of types of lipids or specific formulations containing polymers and lipids. They also never mention Camptotheca alkaloids other than camptothecin. Also, while the formulations disclosed by Liversidge et al. provide a way for maximizing drug delivery capacity, their crystalline nature is problematical because of the well-known phenomenon of crystal growth over time. To overcome crystal growth, nanoparticulate crystals are sometimes coated with crystal growth-inhibiting agents such as nonionic surfactants. In these instances, care must be exerted to insure biocompatibility and nontoxicity of the surfactant or other coating agent.

Another way to improve drug delivery is to formulate medications into nanoparticles. By so doing, for example, hydrophobic or toxic drugs can be more safely delivered. The nanoparticles used for such purposes should be as small as possible, preferably less than 100 nanometers in diameter. Tumors for example, contain leaky blood vessels from which nanoparticles that comprise an antineoplastic medication may extravasate, i.e., the nanoparticles may leak out of the blood vessels into the interstitial space of the tumor tissue.

Collectively, there remains a need in the art for a pharmaceutical formulation that is suitable for administration of a water-insoluble or sparingly water-soluble drugs such as camptothecin or its analogs, wherein (1) the formulation is optimized such that the amount of drug administered is maximized while undesirable side effects are minimized, (2) the rate of drug release can be precisely controlled, (3) no micelles, liposomes, or other vesicles are required, (4) premedication is unnecessary, and (5) the formulation displays excellent stability over extended storage periods.

Among the synthetic analogs and derivatives of camptothecin, 7-ethyl-10-hydroxycamptothecin, designated SN-38, has generated considerable recent interest. It is a metabolite of another synthetic analog, irinotecan, and has demonstrated antineoplastic efficacy somewhat greater that of camptothecin and some of the other analogs. Further, SN-38 has a longer serum half-life than that of natural camptothecin. SN-38 is hydrophobic, and up until now has been difficult to formulate as a drug for therapeutic use in humans. A pro-drug of SN-38, called irinotecan, has been developed for use in humans and is approved for treatment of colon cancer. Unfortunately, irinotecan is associated with adverse side effects, including severe diarrhea. Additionally, irinotecan, as a pro-drug, irinotecan must be converted to the active SN-38 molecule by carboxylesterases in the body. Not all tumors contain sufficient carboxylesterases to form the potent SN-38 drug. It thus appears that SN-38 could be an effective anticancer agent, if it was formulated properly for administration to patients. An improved formulation of SN-38 might also be better tolerated with fewer side effects; in particular, it might be better tolerated in elderly and sick patients, with resulting improvements in efficacy and treatment response.

The instant invention addresses those needs by providing unique formulations of camptothecin analogs that have improved properties useful in drug delivery.

### SUMMARY OF THE INVENTION

It is accordingly a primary object of the invention to address the above-mentioned needs in the art by providing a pharmaceutical formulation effective to deliver a camptothecin analog.

It is another object of the invention to provide a therapeutic method wherein the aforementioned formulation is administered to a patient to treat a condition, disease, or disorder for which the drug is indicated.

Additional objects, advantages, and novel features of the invention will be set forth in part in the description that follows, and in part will become apparent to those skilled in the art upon examination of the following, or may be learned by practice of the invention.

In one aspect of the invention, then, a pharmaceutical formulation is provided that comprises a camptothecin analog, a stabilizing agent that stabilizes the camptothecin analog but does not covalently bind thereto, an optional targeting ligand, an optional secondary stabilizing agent, and an optional excipient. A variety of stabilizing agents may be employed, although polymers, such as poloxamine, poloxamer, polyethylene glycol and poly(ethylene oxide-co-propylene oxide), and/or neutral and/or anionic lipids, such as phospholipids and lecthins, are preferred. The most preferred formulations comprise both polymeric stabilizing agents and lipidic stabilizing agents.

The formulation may be in lyophilized form, which is advantageous for storage stability. The formulation may also be in the form of an aqueous suspension and may further comprise an aqueous vehicle. The aqueous vehicle may be, for example, water, isotonic saline solution, isotonic dextrose or phosphate buffer, and may be instilled with an acoustically active gas to facilitate ultrasound imaging and ultrasonic cavitation for local drug release with ultrasound.

In another aspect of the invention, a method is provided for making the aforementioned formulation, comprising the steps of (1) admixing, in a solvent, a camptothecin analog and a stabilizing agent that stabilizes the camptothecin analog but does not covalently bond thereto; (2) removing the solvent in a manner effective to provide a dry formulation of the camptothecin analog; and (3) rehydrating the dry formulation to form the nanoparticulate formulation. In this method, the solvent may be removed by lyophilization, spray drying, super critical fluid processing or rotary evaporation.

In another aspect of the invention, a method is provided for delivering a drug to a mammalian individual to achieve a desired therapeutic effect, wherein the method involves administering to the individual a therapeutically effective amount of a formulation of the invention, e.g., intravenously, orally, parenterally, intraperitoneally, subcutaneously or via injection into a body cavity such as a joint, or via inhalation for delivery to the lungs.

In a related aspect of the invention, a method is provided for treating an individual suffering from cancer, comprising parenterally administering to the patient a surfactant-free formulation of: (a) drug-containing particles comprised of (i) a stabilizing agent, (ii) a camptothecin analog that complexes with but does not covalently bind to the stabilizing agent, optionally (iii) a targeting ligand, and optionally (iv) an excipient selected from the group consisting of saccharides, liquid polyethylene glycols, propylene glycol, glycerol, ethyl alcohol, and combinations thereof, in (b) an aqueous vehicle.

The present invention is based on the formation of a noncovalent complex of a camptothecin analog with a stabilizing agent. This drug/polymer complex allows for the formation of nanoparticles that may be suspended in an aqueous solution, without requiring chemical modification of the camptothecin analog. This nanoparticle solubilization technology enables the preparation of camptothecin analog formulations with decreased toxicity and improved efficacy. The well-documented problems related to stability, carrier toxicity, and large injection volume of currently available formulations of camptothecin analogs are eliminated with this novel technology.

The noncovalent complex of a camptothecin analog with a stabilizing agent produces a unique class of nanoparticles ranging from about 1 nm to about 500-2000 μm, preferably from about 1 nm to about 500 μm, that can be further treated with a second stabilizing agent to form nanoparticles having diameters ranging from about 1 nm to about 300 nm, preferably from about 20 nm to about 100 nm. The resulting nanoparticles are biocompatible and highly useful for drug delivery. The drug delivery is preferably via intravenous (IV) injection, but the technology has applications for oral, subcutaneous (e.g., sustained release), and pulmonary delivery. For IV delivery, the nanoparticles are useful in that they can, for example, decrease the toxicity of therapeutic agents. Compared to existing methods, larger doses of the camptothecin analogs can therefore be administered intravenously, allowing for higher blood levels of the therapeutic agent, which can yield greater efficacy. For oral applications, the nanoparticles improve the dispersal of camptothecin analogs and increase uptake from the gastrointestinal tract. For sustained release applications, the nanoparticles can be formulated into gels, powders, or suspensions. For pulmonary applications, the nanoparticles' small effective hydrodynamic radii improve the delivery of therapeutic agents into the distal airways, such as the alveoli, thereby allowing systemic delivery of camptothecin analogs via the pulmonary route. In this regard, pulmonary delivery is also useful for local treatment of lung cancer, particularly alveolar cell carcinoma.

### BRIEF DESCRIPTION OF THE DRAWINGS

FIG. 1 is a graph presenting relative tumor size as a function of the time following treatment, for treatment with two formulations of SN-38, irinotecan, and for a non-treated control.

FIG. 2 is a graph presenting relative tumor size as a function of the time following treatment for various lipid stabilized SN-38 formulations of the invention.

### DETAILED DESCRIPTION OF THE INVENTION

A. Definitions and Abbreviations:

Definitions

It is to be understood that unless otherwise indicated, this invention is not limited to specific camptothecin analogs, hydrophilic polymers, phospholipids, excipients, methods of manufacture, or the like, as such may vary. It is also to be understood that the terminology used herein is for the purpose of describing particular embodiments only and is not intended to be limiting.

It must be noted that, as used in the specification and the appended claims, the singular forms “a,” “an,” and “the” include plural referents unless the context clearly dictates otherwise. Thus, for example, reference to “a camptothecin analog” or “a drug” in a formulation means that more than one camptothecin analog can be present, reference to “a hydrophilic polymer” includes combinations of hydrophilic polymers, reference to “a phospholipid” includes mixtures of phospholipids, and the like.

In this specification and in the claims that follow, reference will be made to a number of terms that shall be defined to have the following meanings:

By “pharmaceutically acceptable” is meant a material that is not biologically or otherwise undesirable, i.e., the material may be administered to an individual along with the selected camptothecin analog without causing any undesirable biological effects or interacting in a deleterious manner with any of the other components of the pharmaceutical composition in which it is contained.

“Pharmaceutically or therapeutically effective dose or amount” refers to a dosage level sufficient to induce a desired biological result. That result can be alleviation of the signs, symptoms, or causes of a disease, or any other desired alteration of a biological system.

The term “treat” as in “to treat a disease” is intended to include any means of treating a disease in a mammal, including (1) preventing the disease, i.e., avoiding any clinical symptoms of the disease, (2) inhibiting the disease, that is, arresting the development or progression of clinical symptoms, and/or (3) relieving the disease, i.e., causing regression of clinical symptoms.

The terms “disease,” “disorder,” and “condition” are used interchangeably herein to refer to a physiological state that may be treated using the formulations of the invention.

The number given as the “molecular weight” of a compound, as in the molecular weight of a hydrophilic polymer such as polyethylene glycol, refers to weight average molecular weight M_{w}.

“Lipidic” as used herein refers to a substance that is associated with one or more lipid compounds. Specifically, the choice of the term is used to distinguish it from the more stringently defined terms “liposome” and “micelle”, wherein a liposome implies a vesicular structure with a defined interior aqueous compartment. The arrangement of molecules in a liposome gives rise to a vesicle of at least one lamellar bilayer. Drugs may be sequestered within the interior of liposomes, embedded within the lipid matrix, or affixed to the outside surface of the liposome. In a micelle, there is an arrangement of polar amphipathic molecules, wherein the hydrophilic portion (heads) of the structure defines the exterior surface and the hydrophobic portion (tails) resides interiorly, away from the medium. A micelle is not, by definition, a bilayer, and thus its size and effective carrying capacity is limited according to properties defined by the critical micelle concentration for a given compound. In contrast to liposomes and micelles, lipidic structures are non-liposomal, non-micellar associations of lipid and drug.

The term “lecithin” refers the class of phospholipids called phosphatidylcholines, and generally refers to natural phosphatidylcholines such as dioleylphosphatidylcholine. Such naturally occurring phospholipids are composed of phosphate, choline, glycerol (as the ester), and two fatty acids, and are exclusively modified with phosphatidylcholine at the 3-position of the glycerol. The fatty acyl moieties attached at the 1 and 2 hydroxyl positions of glycerol may be saturated, unsaturated, or a combination of both. Lecithin does not comprise anionic phospholipids such as phosphatidylglycerol, or chemically modified, synthetic phospholipids.

“Nanoparticles” are defined strictly according to size in that they have diameters less than one micrometer. The term may embrace amorphous, structured, or partially crystalline forms.

“Nanocrystals” by contrast are defined as structures with sizes less than one micrometer, but that have at least 99% crystalline structure, regardless of whether the molecular composition of said crystal is purely one component, e.g., drug, or drug in close association with another component.

The term “stabilizer” refers to materials such as lipids and other coating agents, surfactants, or compounds that alter the physical and chemical properties affecting aqueous solubility of a drug when placed in a noncovalent admixture with the drug or drugs.

The “solubility” of a compound refers to its solubility in the indicated liquid determined under standard conditions, e.g., at room temperature (typically about 25° C.), atmospheric pressure, and neutral pH.

In referring to chemical compounds herein, the following definitions apply:

The term “alkyl” refers to a branched or unbranched saturated hydrocarbon group of 1 to 24, typically 10 to 20, carbon atoms, such as methyl, ethyl, n-propyl, isopropyl, n-butyl, isobutyl, t-butyl, octyl, decyl, tetradecyl, hexadecyl, eicosyl, tetracosyl, and the like, as well as cycloalkyl groups such as cyclopentyl, cyclohexyl, and the like.

The term “aryl” refers to an aromatic species containing 1 to 3 aromatic rings, either fused or linked, and either unsubstituted or substituted with one or more substituents. Preferred aryl substituents contain one aromatic ring or two fused aromatic rings.

The term “acyl” refers to a group having the structure R(CO)— wherein R is alkyl or aryl as defined above.

“Optional” or “optionally” means that the subsequently described circumstance may or may not occur, so that the description includes instances where the circumstance occurs and instances where it does not.

Abbreviations

The following abbreviations are used throughout the specification.

| | |
|---|---|
| DOPG | Dioleoyl phosphatidylglycerol; |
| DOPC | Dioleoyl phosphatidylcholine; |
| POPC | Palmitoyl-oleoyl phosphatidylcholine; |
| DLPC | Dilauroyl phosphatidylcholine; |
| DMPC | Dimyristoly phosphatidylcholine; |
| DPPC | Dipalmitoyl phosphatidylcholine; and |
| DSPC | Distearoyl phosphatidylcholine. |

B. Formulations:

The pharmaceutical formulations of the invention are advantageously used to deliver camptothecin analogs by increasing the solubility of the drug in water. The instant invention described herein discloses compositions and methods for making and using preferably noncrystalline, lipidic, and/or polymeric nanoparticles for delivery of camptothecin analogs, preferably SN-38, wherein the nanoparticles are intermediate in size between micelles and liposomes. In some cases, the particles are non-birefringent, indicating amorphous nanoparticles. As the lipid content is decreased, the particles may be birefringent. Because of the way that the particles are made, however, it is believed that when birefringent structures are present, these structures contain substantially more than 0.1% of a second material (e.g., polymer or lipid) interspersed within the drug matrix.

In one embodiment, the formulation represents a unique class of nanoparticles ranging from about 1 nm to about 500-1000 nm, preferably from about 1 nm to about 500 nm, that can be stabilized with a stabilizing agent, preferably lipidic and/or polymeric, to form nanoparticles having diameters ranging from about 1 nm to about 300 nm, preferably from about 20 nm to about 100 nm. The resulting nanoparticles are biocompatible and highly useful for drug delivery. The drug delivery is preferably via IV injection, but the technology has applications for oral, subcutaneous (e.g., sustained release), intracistemal, intranasal, and pulmonary delivery. For IV delivery, the nanoparticles increase the stability and bioavailability of the camptothecin analogs. Compared to existing methods, larger doses of the camptothecin analogs can therefore be administered intravenously, allowing for higher blood levels of the therapeutic agent, which yield greater efficacy.

In the preferred embodiment of the invention, the nanoparticles of the invention are further characterized in that crystal content is substantially under 99%, and generally well under 50%, for most formulations described herein. By this it is meant, that while some lipid or other stabilized formulations show birefringence characteristic of the presence of crystals, the mole fraction of drug in the formulation and the strong noncovalent interaction of lipidic molecules and/or amphipathic polymers with the drug, limit the amount of drug-drug molecular interactions, thus limiting the relative proportion of the drug in crystalline form and the size of the crystallites. These phenomena provide obvious advantages over other formulations described in the art in terms of size stability.

In still another embodiment of the invention, the stabilizing agent may be a combination of a lipidic material and a polymeric material such as poloxamine, poloxamer, or polyethylene glycol in a variety of ratios.

I. The Camptothecin Analog

The drug in the formulation, as noted above, is any camptothecin analog whose systemic bioavailability can be enhanced by increasing the dispersability of the agent in water. It will be appreciated that the invention is particularly useful for delivering camptothecin analogs for which chronic administration may be required, as the present formulations provide for sustained release. The invention thus has the advantage of substantially improving patient compliance, as the potential for missed or mistimed doses is greatly reduced. However, any agent that is typically incorporated into a capsule, tablet, troche, liquid, suspension, or emulsion, wherein administration is on a regular schedule (i.e., daily, more than once daily, every other day, or any other regular interval) can be advantageously delivered using the formulations of the invention.

Camptothecin analogs are topoisomerase inhibitors, such as Camptotheca alkaloids including, but not limited to, homocamptothecin, diflomotecan, exatecan, SN-38, topotecan, irinotecan, and carzelesin, and pharmaceutically acceptable salts of any of the above. Especially preferred are derivatives at the 9 ring position, including 9-nitro-camptothecin and 9-amino-camptothecin; modified 10-position compounds, including 10-hydroxycamptothecin and aminated, aminoalkyl, alkylated, and alkoxylated derivatives of the same; modified 11-position derivatives, including 11-hydroxycamptothecin and aminated, aminoalkyl, alkylated, and alkoxylated derivatives of the same; modified 12-position derivatives, including 12-hydroxycamptothecin and aminated, aminoalkyl, alkylated, and alkoxylated derivatives of the same; 7-position derivatives, including amino, nitro, alkyl, alkylamino, and alkoxy derivatives of the same; and 20 (S) derivatives, including alkylesters and amides of the 20-(OH) group. Preferred compounds within this group include 7-alkylcamptothecin and especially 7-ethyl camptothecin. Other derivatives include permutations of combinations of the above compounds wherein any or all of the 7, 9, 10, 11, and 20(OH) positions may be modified. Among these, a preferred compound and derivative is 7-ethyl-10-hydroxycamptothecin, designated SN-38, a metabolite of irinotecan. One of skill in the art will readily appreciate that any or all functional groups on camptothecin or its analogs are amenable to derivatization into prodrugs, including but not limited to the 9, 10, 11, and 20 ring substituents, with derivatization at the 20 position preferred.

Camptothecin analogs suitable for use in the present formulation may be represented by the structure of formula (I)

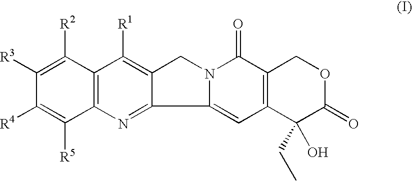

wherein R¹,R², R³,R⁴, and R⁵ are independently selected from the group consisting of H, C₁₋₆ alkyl, C₁₋₆ alkoxy, acyloxy, hydroxyl, sulfhydryl, acyl, halo, amido, C₁₋₆ alkylamido, amino, nitro, and cyano; or R¹ and R² and/or R³ and R⁴ may taken together to form a substituted or unsubstituted five- or six-membered cyclic group containing up to two heteroatoms selected from the group consisting of O, S, and N.

Preferably, R¹, R², R³, R⁴, and R⁵ are independently selected from the group consisting of H, C₁₋₆ alkyl, C₁₋₆ alkoxy, acyloxy, hydroxyl, sulfhydryl, acyl, halo, amido, C₁₋₆ alkylamido, amino, nitro, and cyano. In further preferred embodiments, R₁ is C₁₋₆ alkyl; most preferably, R¹ is methyl. Preferably, R³ is hydroxyl.

In other preferred embodiments, R², R⁴, and R⁵ are H, such that the camptothecin analog has the structure of formula (II)

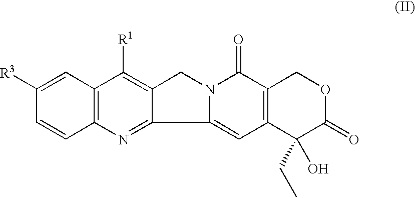

wherein R¹ is C₁₋₆ alkyl and R³ is hydroxyl, sulfhydryl, or amino. Most preferably, R¹ is methyl, R³ is hydroxyl, and the camptothecin analog is 7-ethyl-10-hydroxyl camptothecin, SN-38.

The amount of camptothecin analog in the formulation should be such that the weight ratio of analog to all other components of the formulation is in the range of about 1:1 to 1:100, preferably in the range of about 1:1 to 1:20, more preferably in the range of about 1:2 to 1:20, and optimally about 1:10.

II. The Stabilizing Agent:

The stabilizing agents of the present invention are polymers and/or lipids that are capable of forming noncovalent complexes with the camptothecin analogs. Useful polymers for stabilizing the nanoparticles include linear or branched polyethylene glycol (PEG), and copolymers of PEG with polypropylene oxide, such as the PLURONICS® (BASF Corporation, Mount Olive, N.Y.). Particularly preferred polymers are poloxamer, a block copolymer of propylene oxide flanked on each end by ethylene oxide; and poloxamine, a polyalkoxylated symmetrical block polymer of ethylene diamine conforming to the general type [(PEG)ₓ-(PPG)_{Y}]₂—NCH₂CH₂N—[(PPG)_{Y}-(PEG)_{X}]₂. Preferred species of poloxamer are the PLURONICS®, with PLURONIC® F68 being highly preferred. Suitable poloxamines include the TETRONICS® with TETRONIC® 908 is a preferred species with a molecular weight of 25,000 Daltons. Other derivatives with shorter PEG and PPG copolymeric chains having molecular weights between 1650 to 25 kDaltons are also suitable.

Useful lipids include phospholipids and lecithins. Suitable phospholipids for the invention include but are not limited to diacyl derivatives of phophatidylcholine, phosphatidylethanolamine, phosphatidylserine, phosphatidylglycerol, and phosphatidic acid. The fatty acyl chain may be from 10 to 22 carbons in length and may be saturated, monounsaturated, or polyunsaturated. The fatty acid at the 1 and 2 positions may be mixed or the same in the acylglyceryl moieties. Preferred saturated fatty acyl moieties include lauryl, myristyl, palmityl, stearyl, or higher chain derivatives; preferred unsaturated acyl moieties include oleyl chains. A given phospholipid may contain two identical chains, as in DOPE (dioleylphosphatidyl ethanolamine), or mixed chains as in POPE (1-palmitoyl-2-oleylphosphatidylethanolamine).

Where present, lipids conjugated with polyethylene glycol (PEG) may utilize polymer lengths of PEG ranging from 350-7000 Daltons, and preferably from 750 to 5,000 Daltons. PEG chains may be either linear or branched and may be derivatized with amino, carboxyl, acyl, or sulfonyl ends.

Examples of suitable lipids include, but are not limited to, phosphatidylglycerol, phosphatidic acid, phosphatidylserine, phosphatidylinositol, cerebrosides, gangliosides, sphingosines, cardiolipin, and sulfatides. Particularly preferred lipids include, but are not limited to, dioleoyl phosphatidylglycerol (DOPG), dioleoyl phosphatidylcholine (DOPC), palmitoyl-oleoyl phosphatidylcholine (POPC), dilauroyl phosphatidylcholine (DLPC), dimyristoly phosphatidylcholine (DMPC), dipalmitoyl phosphatidylcholine (DPPC), distearoyl phosphatidylcholine (DSPC), and phosphatidylglycerol. A particularly preferred lipid is palmitoyloleylphosphatidylglycerol (POPG).

When an lipid is employed as the stabilizing agent in the formulation, the amount of lipid may range from about 0.1% by weight up to about 99% of the formulation. More preferably the lipid will range from about 1% to about 90% by weight and still more preferably from about 2% to about 50% by weight. Preferred ratios of drug to lipid range from 1:1 to approximately 1:50, with ranges of 1:10 to 1:30 being preferred.

When a polymer is used as the stabilizing agent in the formulation of the invention, the polymer, e.g. poloxamer or poloxamine, is generally formulated with the active drug, e.g. SN-38, in a weight ratio from about 0.1% by weight polymer up to about 99% by weight polymer, with the drug ranging from between 99.9% by weight to about 0.1% by weight. Preferably, the polymer ranges from about 10 to about 90% by weight. More preferably, the polymer ranges from about 10 to about 50% by weight relative to the amount of drug. Most preferably, the polymer is about 50% by weight relative to the drug.

Examples of suitable polymeric stabilizing agents include, but are not limited to, polyethylene glycol, polypropylene glycol, polyvinyl alcohol, polyvinyl pyrrolidone, polylactide, poly(lactide-co-glycolide), polysorbate, polyethylene oxide, polypropylene oxide, poly(ethylene oxide-co-propylene oxide), poly(oxyethylated) glycerol, poly(oxyethylated) sorbitol, poly(oxyethylated) glucose), and derivatives, mixtures, and copolymers thereof. Examples of suitable derivatives include those in which one or more C—H bonds, e.g., in alkylene linking groups, are replaced with C—F bonds, such that the polymers are fluorinated or even perfluorinated. The polymer may be linear or branched. When branched polymers are used, they contain between about 3 and 20 arms, more preferably between 4 and 8 arms and most preferably 4 arms. When branched polymers are used, these preferably contain an inner hydrophobic core and outer hydrophilic arms (e.g. inner arms made of polylactide and outer arms made of polyethyleneglycol or polypropyleneglycol).

As discussed above, one preferred polymer for use as a stabilizing agent in the present formulations is polyethylene glycol (PEG) or a copolymer thereof, e.g., polyethylene glycol containing some fraction of other monomer units (e.g., other alkylene oxide segments such as propylene oxide), with polyethylene glycol itself most preferred. The polyethylene glycol used may be either branched PEG (including “dendrimeric” PEG, i.e., higher molecular weight, highly branched PEG) or star PEG, optionally conjugated to a phospholipid moiety as will be discussed below. Covalent conjugates of linear PEG and phospholipids may also be used. Combinations of different types of PEG (e.g., branched PEG and linear PEG, star PEG and linear PEG, branched PEG and phospholipid-conjugated linear PEG, etc.) may also be employed.

Branched PEG molecules will generally although not necessarily have a molecular weight in the range of approximately 1,000 to 600,000 Daltons, more typically in the range of approximately 2,000 to 10,000 Daltons, preferably in the range of approximately 5,000 to 40,000 Daltons. Branched PEG is commercially available, such as from Nippon Oil and Fat (NOF Corporation, Tokyo, Japan) and from Shearwater Polymers (Huntsville, Ala.), or may be readily synthesized by polymerizing lower molecular weight linear PEG molecules (i.e., PEG 2000 or smaller) functionalized at one or both termini with a reactive group. For example, branched PEG can be synthesized by solution polymerization of lower molecular weight PEG acrylates (i.e., PEG molecules in which a terminal hydroxyl group is replaced by an acrylate functionality —O—(CO)—CH═CH₂) or methacrylates (similarly, PEG molecules in which a hydroxyl group is replaced by a methacrylate functionality —O—(CO)—C(CH₃)═CH₂) in the presence of a free radical polymerization initiator such as 2,2′-azobisisobutyronitrile (AIBN). If desired, mixtures of PEG monoacrylates or monomethacrylates having different molecular weights can be used in order to synthesize a branched polymer having “branches” or “arms” of differing lengths. Branched PEGs have 2 or more arms but may have as many as 1,000 arms. The branched PEGs herein preferably have about 4 to 40 arms, more preferably about 4 to 10 arms, and most preferably about 4 to 8 arms. Higher molecular weight, highly branched PEG, e.g., branched PEG having a molecular weight of greater than about 10,000 and at least about 1 arm (i.e., one branch point) per 5,000 Daltons, will sometimes be referred to herein as “dendrimeric” PEG. Such PEG is preferably formed by reaction of a hydroxyl-substituted amine such as triethanolamine with lower molecular weight PEG that may be linear, branched, or star, to form a molecular lattice that serves as the stabilized matrix and entraps the camptothecin analog to be delivered. Dendrimeric structures, including dendrimeric PEG, are described, inter alia, by Liu et al. (1999) PSTT 2(10):393-401.

Star molecules of PEG are available commercially (e.g., from Shearwater Polymers, Huntsville, Ala.) or may be readily synthesized using living free radical polymerization techniques as described, for example, by Gnanou et al. (1988) Makromol. Chem. 189:2885-2892 and Desai et al., U.S. Pat. No. 5,648,506. Star PEG generally has a central core of divinyl benzene or glycerol. Preferred molecular weights for star molecules of PEG useful herein are typically in the range of about 1,000 to 500,000 Daltons, although molecular weights in the range of about 10,000 to 200,000 are preferred.

As explained above, conjugates of polymers and phospholipids, particularly PEG-phospholipid conjugates (also termed “PEGylated” phospholipids), are also useful in the present formulations. The polyethylene glycol in the PEGylated phospholipids may be branched, star, or linear. Conjugates of linear PEG and phospholipids, if used, will generally although not necessarily employ PEG have a molecular weight in the range of approximately 100 to 50,000 Daltons, preferably in the range of approximately 350 to 40,000 Daltons. It will be appreciated by those skilled in the art that the aforementioned molecular weight ranges correspond to a polymer containing approximately 2 to 1,000 ethylene oxide units, preferably about 8 to 800 ethylene oxide units. The phospholipid moiety that is conjugated to the PEG may be anionic, neutral, or cationic, of naturally occurring or synthetic origin, and normally comprises a diacyl phosphatidylcholine, a diacyl phosphatidylethanolamine, a diacyl phosphatidylserine, a diacyl phosphatidylinositol, a diacyl phosphatidylglycerol, or a diacyl phosphatidic acid, wherein each acyl moiety can be saturated or unsaturated and will generally be in the range of about 10 to 22 carbon atoms in length. Preferred compounds are polymer-conjugated diacyl phosphatidyl-ethanolamines having the structure of formula (III)

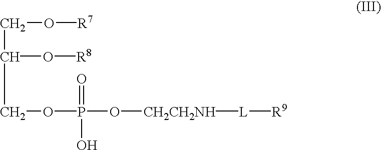

wherein R⁷ and R⁸ are the acyl groups, R⁹ represents the hydrophilic polymer, e.g., a polyalkylene oxide moiety such as PEG, poly(ethylene oxide), poly(propylene oxide), poly(ethylene oxide-co-propylene oxide), or the like (for linear PEG, R⁹ is —O—(CH₂CH₂O)ₙ—H), and L is an organic linking moiety such as a carbamate, an ester, or a diketone having the structure of formula (IV)

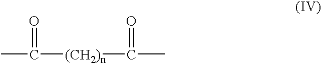

wherein n is 1, 2, 3, or 4. Preferred unsaturated acyl moieties are esters formed from oleic and linoleic acids, and preferred saturated acyl moieties are palmitate, myristate, and stearate. Particularly preferred phospholipids for conjugation to linear, branched, or star PEG herein are dipalmitoyl phosphatidylethanolamine (DPPE) and 1-palmitoyl-2-oleyl phosphatidylethanolamine (POPE).

The conjugates may be synthesized using art-known methods such as described, for example, in U.S. Pat. No. 4,534,899 to Sears. That is, synthesis of a PEG-phospholipid conjugate or a conjugate of a phospholipid and an alternative hydrophilic polymer may be carried out by activating the polymer to prepare an activated derivative thereof, which has a functional group suitable for reaction with an alcohol, a phosphate group, a carboxylic acid, an amino group, or the like. For example, a polyalkylene oxide such as PEG may be activated by the addition of a cyclic polyacid, particularly an anhydride such as succinic or glutaric anhydride (ultimately resulting in the linker of formula (II) wherein n is 2 or 3, respectively). The activated polymer may then be covalently coupled to the selected phosphatidylalkanolamine, such as phosphatidylethanolamine, to give the desired conjugate.

The polymer may be modified in one or more ways. For drugs that are ionized at physiological pH, charged groups may be inserted into the hydrophilic polymer in order to modify the sustained release profile of the formulation. To reduce the rate of drug release and thereby provide sustained delivery over a longer time period, negatively charged groups such as phosphates and carboxylates are used for cationic drugs, whereas positively charged groups such as quaternary ammonium groups are used in combination with anionic drugs. A terminal hydroxyl group of a hydrophilic polymer such as PEG may be converted to a carboxylic acid or phosphate moiety by using a mild oxidizing agent such as chromic (VI) acid, nitric acid, or potassium permanganate; a preferred oxidizing agent is molecular oxygen used in conjunction with a platinum catalyst. Introduction of phosphate groups may be carried out using a phosphorylating reagent such as phosphorous oxychloride (POCl₃). Terminal quaternary ammonium salts may be synthesized, for example, by reaction with a moiety such as

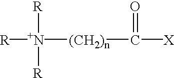

wherein R is H or lower alkyl (e.g., methyl or ethyl), n is typically 1 to 4, and X is an activating group such as Br, Cl, I, or an —NHS ester. If desired, such charged polymers may be used to form higher molecular weight aggregates by reaction with a polyvalent counter ion.

Other possible modifications to the polymer include, but are not limited to, the following. A terminal hydroxyl group of a PEG molecule may be replaced by a thiol group using conventional means, e.g., reacting hydroxyl-containing PEG with a sulfur-containing amino acid such as cysteine, using a protected and activated amino acid. Such “PEG-SH” is also commercially available, for example from Shearwater Polymers. Alternatively, a mono(lower alkoxy)-substituted PEG such as monomethoxy polyethylene glycol (MPEG) may be used instead of polyethylene glycol per se, so that the polymer terminates with a lower alkoxy substituent (such as a methoxy group) rather than with a hydroxyl group. Similarly, PEG amine may be used in lieu of PEG so that a terminal amine moiety —NH₂ is present instead of a terminal hydroxyl group.

In addition, as discussed above, the polymer may contain two or more types of monomers, as in a copolymer wherein propylene oxide groups (—CH₂CH₂CH₂O—) have been substituted for some fraction of ethylene oxide groups (—CH₂CH₂O—) in polyethylene glycol. Incorporating propylene oxide groups will tend to increase the stability of the stabilized camptothecin analog complex, thus decreasing the rate at which the drug is released in the body. The larger the fraction of propylene oxide blocks, the slower the drug release rate.

The polymer may also contain hydrolyzable linkages to enable hydrolytic degradation within the body, and thus facilitate drug release from the polymeric matrix. Suitable hydrolyzable linkages include any intramolecular bonds that can be cleaved by hydrolysis, typically in the presence of acid or base. Examples of hydrolyzable linkages include, but are not limited to, those disclosed in International Patent Publication No. WO 99/22770 to Harris, such as carboxylate esters, phosphate esters, acetals, imines, ortho esters, and amides.

Other suitable hydrolyzable linkages include, for example, enol ethers, diketene acetals, ketals, anhydrides, and cyclic diketenes. Formation of such hydrolyzable linkages within the hydrophilic polymer is conducted using routine chemistry known to those skilled in the art of organic synthesis and/or described in the pertinent texts and literature. For example, carboxylate linkages may be synthesized by reaction of a carboxylic acid with an alcohol, phosphate ester linkages may be synthesized by reaction of a phosphate group with an alcohol, acetal linkages may be synthesized by reaction of an aldehyde and an alcohol, and the like. Thus, polyethylene glycol containing hydrolyzable linkages “X” might have the structure -PEG-X-PEG- or alternatively might be a matrix having the structure

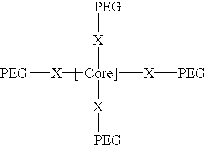

wherein the core is a hydrophobic molecule such as pentaerythritol. Such polymers may be synthesized by reaction of various -PEG-Y molecules with -Core-Z or PEG-Z molecules wherein Z and Y represent groups located at the terminus of individual PEG molecules and are capable of reacting with each other to form the hydrolyzable linkage X.

Accordingly, it will be appreciated that the rate of drug release from the stabilized camptothecin analog matrix can be controlled by adjusting the degree of branching of the polymer, by incorporating different types of monomer units in the polymer structure, by functionalizing the hydrophilic polymer with different terminal species (which may or may not be charged), and/or by varying the density of hydrolyzable linkages present within the polymeric structure.

As illustrated above, the branched PEG molecule may be modified to have a hydrophobic core. For example, if the central core is pentaerythritol, the innermost arms bound to the pentaerythritol may comprise a polymer more hydrophobic than PEG. Useful polymers to accomplish this include polypropylene glycol and polybutylene glycol. Useful monomers for constructing the inner, hydrophobic core structures of the arms include propylene oxide, butylene oxide, and copolymers of the two; and lactic acid and copolymers of lactic acid with glycolide (polylactide-co-glycolide and copolymers of the foregoing with polyethylene glycol). The preferred materials for constructing an inner hydrophobic core include polypropylene glycol and copolymers of propylene oxide with ethylene oxide. Useful polymers for constructing the outer, peripheral parts of the arms include polyethylene glycol, polysialic acid, and other hydrophilic polymers, with PEG most preferred. It is possible that a fraction of the monomers in the outer portion of a given arm of the carrier molecule may be replaced with PEG, but in this case, there will be substantially more of the hydrophilic monomer (e.g., ethylene oxide) than the hydrophobic monomer (e.g., propylene oxide).

The relative proportion of hydrophobic polymer within the branched polymer may vary from about 10 wt. % to about 90 wt. % on a weight/weight ratio, preferably from about 40 wt. % to about 60 wt. %. When more hydrophobic polymer is used, the drug loading capacity of the branched molecule may be increased for hydrophobic drugs. A most preferred ratio is about 50 wt. % of hydrophobic polymer, e.g., polypropylene glycol, and 50 wt. % of hydrophilic polymer (e.g., PEG) in the outer arms.

The branched molecules comprising a hydrophobic core and peripheral hydrophilic arms are thought to have a number of advantages for drug delivery. The hydrophobic core may better stabilize hydrophobic drugs within the branched molecule and, as the drug is stabilized within the core, the free arms of the PEG may be better able to maintain a random state in which the PEG molecules move freely within solution. The outer, hydrophilic PEG layer may act as a steric barrier, inhibiting or decreasing the aggregation of individual branched molecules into particles. Additionally, in instances when targeting ligands are attached to the termini of the peripheral hydrophilic arms, targeting is facilitated by the unencumbered and exposed nature of the outer PEG arms. As will be discussed further on, a wide variety of targeting ligands can be covalently bound to the free ends of the PEG. The hydrophobic and hydrophilic components of the arms may be linked together by a variety of different linkers. Such linkers include ethers, amides, esters, carbamates, thioesters, and disulfide bonds. In general, the linker employed is used to attain the desired drug delivery properties of the pharmaceutical formulation. Metabolizable bonds can be selected to improve excretion of the carrier molecule as well as to improve drug release.

As previously mentioned, when branched PEG polymers are used as the stabilizing agent, the free ends of the branches can be substituted with one or more targeting ligands per carrier molecule. More than one kind of targeting ligand may be bound to each carrier molecule to facilitate binding to a target cell bearing more than one kind of receptor. A wide variety of ligands may be used in this regard. Exemplary targeting ligands include, for example, proteins, peptides, polypeptides, antibodies, antibody fragments, glycoproteins, carbohydrates, hormones, hormone analogs, lectins, amino acids, sugars, saccharides, vitamins, steroids, steroid analogs, enzyme cofactors, biocamptothecin analogs, and genetic material. Suitable targeting ligands and methods of synthesizing and attaching such ligands are discussed in commonly assigned U.S. patent application Ser. No. 09/478,124, filed Jan. 5, 2000, entitled, “Pharmaceutical Formulations For The Delivery Of Drugs Having Low Aqueous Solubility.”

In another embodiment, the stabilizing agent contains a mixture of polymeric stabilizing agents and lipidic stabilizing agents. For example, a particularly preferred formulation might contain about 2 parts drug, 1 part poloxamer, and 8 parts by weight of phosphatidylglycerol, wherein the drug is SN-38. Preferred ranges for the ratio of drug to lipid to polymeric component when combinations of stabilizing agents are used range between approximately 1:2:1 to approximately 1:20:5, most preferably from approximately 1:5:1 to approximately 1:10:2. In embodiments where lipids and polymers are both used as the stabilizing agent, the polymeric component of the stabilizing agent is generally added during rehydration, as will be discussed below.

Secondary stabilizing agents may also be added to the formulation and are useful for reducing particle size. Preferably, the secondary stabilizing agent acts to stabilize the surface of the complex by virtue of a combination of hydrophilic and hydrophobic interactions. Thus, it is preferred that the secondary stabilizing agent polymer contains both hydrophilic and hydrophobic groups or domains, thus allowing the combination of interactions to occur. It is also preferred that the secondary stabilizing agent contain a sufficient amount of hydrophilic surface area so that post-stabilization nanoparticles remain suspended within an aqueous solution and avoid clumping.

An exemplary secondary stabilizing agent is a polymer having a molecular weight ranging from about 400 Daltons to about 400,000 Daltons, more preferably from about 1,000 Daltons to about 200,000 Daltons, and still more preferably from about 3,000 Daltons to about 100,000 Daltons. The secondary stabilizing agent may be derived from natural, recombinant, synthetic, or semisynthetic sources. Most preferably, the secondary stabilizing agent will be a protein or a peptide. Useful preferred proteins include albumin, collagen, fibrin, immunoglobulins, hemoglobin, vascular endothelial growth factor, vascular permeability factor, epidermal growth factor, fibroblast growth factor, fibronectin, vitronectin, and cytokines such as interleukins (e.g., IL-3 and IL-12).

Suitable secondary stabilizing proteins include, but are not limited to: serum proteins, i.e., albumin (especially recombinant and defatted), arnylins, atrial natriuretic peptides, endothelins and endothelin inhibitors, urokinase, streptokinase, staphylokinase, vasoactive intestinal peptide, HDL, LDL, VLDL, etc.; agglutination (antihemophilia) factors (e.g., Factor VIII, Factor IX, and subtypes thereof), decorsin, serum thymic factor, etc.; peptide hormones, e.g., ACTH, FSH, LH, thyroxin, insulin, vasopressin, bradykinin and bradykinin potentiators, HGH, CRF (corticotropin releasing factor), oxytocin, gastrins, LH-RH, MSH (melanocyte stimulating hormone) and MSH releasing factor; parathyroid hormones and analogs; pituitary adenylate cyclase activating polypeptide; secretins; thyrotropin releasing hormone, etc.; structural proteins, e.g., collagens, amyloid proteins, brain natriuretic peptides, elafin, fibronectin and fibronectin fragments, laminin, sarafotoxins, etc.; growth factors, e.g., nerve growth factor, platelet derived growth factor, epidermal growth factor, vascular endothelial growth factor, tumor necrosis factor, CINC-I (cytokine-induced neutrophil chemoattractant), growth hormone releasing factor, liver cell growth factor, midkines, neurokinins, neuromedins, etc.; metabolic potentiators, e.g., erythropoietin, adrenomedullin and adrenomedullin antagonists, o-agatoxin TK, agelenin, angiotensins, calcicludine, calciseptine, calcitonin and calcitonin antagonists, calmodulin, charybdotoxin, chlorotoxin, conotoxins, endorphins, neo-endorphins, glucagon and variants, guanylins, iberiotoxin, kaliotoxin, margatoxin, mast cell degranulating peptide, neurotensins, pancreastatins, PLTX-11, scylotoxin, ATPase inhibitors, somatostatins, somatomedin, uroguanylin, etc.; nuclear binding proteins, e.g., histones, spermine, spermidine, nuclear localization sequences, telomerase, etc.; enzymes, e.g., cholecystokinin, cathepsins, etc.; antivirals, i.e., IFN-α, IFN-β, IFN-γ, virus replication inhibiting peptide, etc.; immunoglobulins, i.e., IgA, IgD, IgE, IgG, IgH, and subtypes; and miscellaneous proteins such as apamin, bombesin, casomorphins, conantokins, defensin-1, dynorphins, enkephalins, galanins, magainin, nociceptin, osteocalcins, substance P, xenin, etc. While not wishing to be limited to the preceding examples, one of skill in the art will recognize that the examples given may be used individually or in combination.

The secondary stabilizing protein may also serve as a targeting agent or binding ligand to direct the nanoparticles and drugs therein to a certain site. The preferred protein is albumin, in particular human serum albumin, and even more preferably recombinant derived human albumin. Another preferred protein is defatted albumin, either native or recombinant. For veterinary applications, the albumin is preferably from the patient's species. The stabilizing albumin is generally added to the nanoparticles at an effective stabilizing concentration, generally in the range of about 0.001% w/v to about 10% w/v, preferably in the range of about 0.01% to about 5%, more preferably in the range of about 0.1% to about 2.5%, and most preferably in the range of about 0.25% to about 1.5%. Note that more than one protein may be used to stabilize the nanoparticles. For example, the particles may be formulated with about 1.0% w/v albumin and about 0.1% w/v EGF. In this case, the EGF serves as a targeting ligand to help the nanoparticle bind to tissues with increased expression of the EGF receptor.

The protein may be naturally occurring, a protein fragment (e.g., a fragment of the gamma-carboxy terminus of fibrinogen), or chemically modified. For example, albumin or other proteins may be modified with one or more hydrophilic or targeting moieties. The protein, for example, may be modified by binding one or more PEG residues per protein molecule, typically between 1 and 100 PEG residues per protein molecule, but more preferably between 1 and 10 PEG residues. For example, mono or bifunctional PEG groups may be coupled to the protein through linkages such as ethers or biodegradable bonds such as esters, amides, carbamates, thioesters, disulfides, thiocarbamates, phosphate esters, and phosphoamides. The resulting “PEGylated” protein enables the protein to stabilize the surface of the nanoparticle while the PEG groups help to protect the nanoparticle surface from nonspecific interaction with serum proteins. In this manner, the “PEGylated” proteins increase the serum half-lives of the nanoparticles.

In addition to the proteins enumerated above, the secondary stabilizing polymers may be other natural polymers, such as: cellulose and dextran; semi-synthetic cellulose derivatives such as methylcellulose and carboxymethylcellulose; and synthetic polymers such as polyinylalcohol polyvinylpyrrolidone and copolymers containing PEG and a second polymer such as polypropylene glycol (PPG) (e.g. those available under the Pluronic trademark). Synthetic polymers such as the PLURONICS®, i.e. copolymers of PEG and PPG, may be incorporated into mixtures of secondary stabilizing agents, e.g., with albumin. Preferred block copolymers include, but are not limited to, polyethylene glycol-N-carboxyanhydride of 6-(benzyloxycarbonyl)-1-lysine, polyethylene glycol-poly-1-lysine, and polyethylene glycol-polyaspartic acid. Methods for synthesizing the above copolymers are described in detail by Harada and Kataoka (1995) Macromolecules 28:5294-5299. One of skill in the art will readily recognize that the same synthetic methods can be used to substitute polypropylene glycols for PEG to make the PPG block copolymer analogs of the above.

Other Components of the Formulation

Other moieties may be incorporated into the present formulations as excipients in order to reduce the particle size of the stabilized camptothecin analog matrix. For intravenous administration in particular, particle size is critical, and is generally in the range of about 1 nm to 10 μm, preferably in the range of about 5 nm to 500 nm, and most preferably in the range of about 30 nm to 250 nm (the values given are number average).

Compounds other than the stabilizing agents are also useful for reducing particle size; these other compounds include, but are not limited to, cholic acids, cholic acid salts, saccharides (such as sorbitol, sucrose, and trehalose), polyhydroxyalcohols (such as glycerol), and liquid polyethylene glycols (i.e., PEG having a molecular weight less than about 1,000 Daltons). The formulations of the invention can also contain pharmaceutically acceptable auxiliary agents as required in order to approximate physiological conditions; such auxiliary agents include pH adjusting and buffering agents, tonicity adjusting agents, and the like. Lipid-protecting agents that serve to minimize free radical and peroxidative damage upon storage may also be advantageous. Suitable lipid protective agents include alpha-tocopherol, ethylenediaminetetraacetic acid (EDTA) and water-soluble, iron-specific chelators such as deferoxamine. Additionally, for lyophilized compositions that are to be hydrated prior to use, it may be desirable to include one or more cryoprotectants or antiflocculants in order to facilitate rehydration and formation of a substantially homogeneous suspension. For compositions that are to be stored in liquid form, it is preferred that one or more conventional antibacterial agents be included. Still other additives that may be incorporated into the present formulations include radioactive or fluorescent markers useful for imaging purposes. Radioactive markers include, for example, technetium-99 and indium-11, while an exemplary fluorescent marker is fluorescein. The excipients can be included in an amount up to about 50 wt. % of the formulation, but preferably represent less than about 10 wt. % of the formulation.

Generally, any additional components to the formulation are added to the complex in an aqueous medium. The complex and additional components are then subjected to a mechanical dispersal process that helps to break the complex into nanoparticles stabilized by the stabilizing agent and incorporating the additional components. Useful mechanical dispersal processes include shaking, agitation (e.g., vortexing), sonication, extrusion under pressure, microfluidization, microemulsification, and high speed blending.

Manufacture and Storage

The formulations of the invention are manufactured using standard techniques and reagents known to those skilled in the art of pharmaceutical formulation and drug delivery and/or described in the pertinent texts and literature. See Remington: The Science and Practice of Pharmacy, 19th Ed. (Easton, Pa.: Mack Publishing Co., 1995), which discloses conventional methods of preparing pharmaceutical compositions that may be used as described or modified to prepare pharmaceutical formulations of the invention. In a preferred embodiment, the stabilizing agent and camptothecin analog are mixed together in an organic solvent or solvent system such as isopropanol, t-butanol, benzene/methanol, ethanol, or an alternative suitable solvent as will be apparent to those of skill in the art, and then lyophilized.

The lyophilized mixture is then rehydrated with a rehydration solution that may contain an additional component of the stabilizing agent. In embodiments where the stabilizing agent comprises a lipidic stabilizing agent and a polymeric stabilizing agent, the lipidic component is first mixed with the camptothecin analog and freeze-dried, and the polymeric component is added to the formulation during the rehydration step. Additional components such as secondary stabilizing agents, e.g., proteins, excipients, and targeting ligands, may also be incorporated into the formulation during rehydration.

It is significant to note that the present method of manufacturing the stabilized analog does not require extensive preprocessing such as grinding or milling of either the stabilizing agent or the drug. Also, heat is not required in order to melt the stabilizing agent or the drug, although heat may be applied in order to facilitate dissolution of the stabilizing agent into the solvent. This ability to form the stabilized camptothecin analog without melting or other preprocessing is a significant advantage of the method of the invention, as it reduces both manufacturing time and cost.

Although lyophilization is the preferred method for solvent removal, the solvent may also be removed by subjecting the mixture to rotary evaporation to yield a powder or a solid matrix. When a solid matrix is obtained, the material may be ground via ball milling or subjected to other mechanical shear stress to achieve a finely ground powder of nanoparticulate material. The resulting nanoparticles may be additionally stabilized with surfactants, phospholipids, stabilizing agents including albumin, and other stabilizing materials, as discussed above.

Another method of manufacturing the formulation is spray drying. In this method, a suitable organic solvent, ideally having a flash point sufficiently above the drying temperature, is used. Formulations made using this method are in the form of a fluffy, dry powder. Alternatively, the components of the final product may be dissolved in a supercritical fluid such as compressed carbon dioxide, and then ejected under pressure and shearing force to form dried particles of the drug-containing formulation.

The formulation is preferably stored in lyophilized form, in which case the lyophilized composition is rehydrated prior to use. Rehydration is carried out by mixing the lyophilized composition with an aqueous liquid (e.g., water, isotonic saline solution, phosphate buffer, etc.) to provide a total solute concentration in the range of about 10 to 100 mg/mL and a drug concentration in the range of about 0.02 to 20 mg/mL, preferably about 0.5 to 10 mg/mL. The formulation may, however, be stored in the aqueous state, e.g., in pre-filled syringes or vials. The formulation may also be stored as a liquid in a physiologically acceptable organic solvent such as ethanol, propylene glycol, or glycerol, to be diluted with water prior to injection into a patient. The lyophilized and rehydrated formulations may be stored at various temperatures, such as at freezing conditions (below about 0° C. and as low as about −40° C. to −100° C.), refrigerated conditions generally between about 0° C. and 15° C., room temperature conditions generally between about 15° C. and 28° C., or at elevated temperatures as high as about 40° C.

The particle size of individual particles within the formulation will vary, depending upon the molecular weight and concentration of the stabilizing agent, the amount of camptothecin analog as well as its solubility profile (i.e., its solubility in water and the hydrophilic polymer), the use of secondary stabilizing agents, and the conditions used in manufacturing. That is, as noted in the preceding section, secondary stabilizing agents and various excipients may be used to facilitate rehydration and provide a substantially homogeneous dispersion. Additionally, mechanical processing techniques can be used to adjust particle size to the appropriate diameter for the intended application; for example, after rehydration, the formulation can be subjected to shear forces with microfluidization, sonication, extrusion, or the like.

Formulations made with stabilizing agents can have a particle size on the order of about 20 nm to 100 nm. These smaller particles, by virtue of their larger accessible surface-to-volume ratio, tend to release drug quite rapidly, while larger particles, e.g., over 10 μm in diameter, will provide for far more gradual, sustained release of drug. The preferred particle size herein is in the range of about 1 nm to 500-1000 μm in diameter. For intramuscular and subcutaneous injection, the particle size should be in the range of about 1 nm to 500 μm, preferably in the range of about 10 nm to 300 μm, and most preferably in the range of about 20 μm to 200 μm. For intravenous administration, as noted previously, particle size is optimally in the range of about 30 nm to 250 nm. For interstitial administration and fracture or wound packing, particle size can be up to 1,000 μm, while for embolization, particle size will generally be between about 100 μm and 250 μm.

The formulation can be sterilized using heat, ionizing radiation, or filtration. For drugs that are thermally stable, heat sterilization is preferable. Lower viscosity formulations can be filter-sterilized, in which case the particle size should be under about 200 nm. Aseptic manufacturing conditions may be employed as well, and lyophilization is also helpful to maintain sterility and ensure a long shelf life. In addition, as noted in the preceding section, antibacterial agents may be included in aqueous formulations in order to prevent bacterial contamination.

Typical formulations of the invention are presented in Tables 1, 2, and 3 below. In Table 1 the drug is SN-38 and the stabilizers are saturated or unsaturated lipids. In Table 2 the drug is SN-38 and the stabilizers are a combination of lipid, poloxamine, and branched polyethylene glycols (bPEG) in various ratios. Table 3 illustrates various lipid components wherein at least one component is a PEGylated lipid.

TABLE 1

| | | | COMPOSITION | SIZE OF | |
|---|---|---|---|---|---|
| ID# | COMPONENT 1 | COMPONENT 2 | RATIO* | MAJOR PEAKS | 24-HR STABILITY |
| 1 | DOPG | — | 1:15 | 67.1 nm, 260.4 nm | slight settling |
| 2 | DOPC | DOPG | 1:5:10 | 65.4 nm | slight settling |
| 3 | DOPC | DOPG | 1:10:5 | 21.3 nm | slight settling |
| 4 | DOPC | — | 1:15 | 26.3 nm | slight settling; phases |
| | | | | | separate |
| 5 | POPC | — | 1:15 | 27.2 nm | slight settling; phases |
| | | | | | separate |
| 6 | DLPC | — | 1:17 | 194.9 nm, 1050 nm | slight settling; phases |
| | | | | | separate |
| 7 | DMPC | — | 1:15 | 29.6 nm | slight settling; phases |
| | | | | | separate |
| 8 | DPPC | — | 1:15 | 2215 nm | definite phase |
| | | | | | separation |
| 9 | DSPC | — | 1:15 | >3000 nm | definite phase |
| | | | | | separation |
| 10 | POPC | — | 1:30 | 20.3 nm | slight settling; phases |
| | | | | | separate |
| 11 | POPC | — | 1:15 | 26.4 nm | slight settling; phases |
| | | | | | separate |
| 12 | POPC | — | 2:15 | 37.0 nm, 80.4 nm | slight settling; phases |
| | | | | | separate |
| 13 | POPC | — | 4:15 | 504.9 nm, 2291 nm | slight settling; phases |
| | | | | | separate |
| 14 | POPC | DOPG | 1:18:2 | 22.1 nm | transparent |
| 15 | POPC | DOPG | 1:16:4 | 9.1 nm | transparent |
| 16 | POPC | DOPG | 1:19:1 | 34.2 nm | transparent |

*w/w ratio, wherein the first component is SN-38, and the subsequent components are the lipid component #1 and #2, respectively.
In cases of settling or phase separation, the phases are easily resuspended.

TABLE 2

| | | | COMPOSITION | SIZE OF | |
|---|---|---|---|---|---|
| ID# | COMPONENT 1 | COMPONENT 2 | RATIO* | MAJOR PEAKS | 24-HR STABILITY |
| 17 | 10k bPEG | DOPG | 1:50:4 | 71.5 nm, 395 nm | definite phase |
| | | | | | separation |
| 18 | POPC | 10k bPEG | 1:20:8 | 25.6 nm | definite phase |
| | | | | | separation |
| 19 | POPC | 10k bPEG | 1:20:4 | 24.9 nm | definite phase |
| | | | | | separation |
| 20 | POPC | 10k bPEG | 1:20:2 | 25.0 nm | definite phase |
| | | | | | separation |
| 21 | POPC | DOPG/10k | 1:20:2:8 | 19.4 nm | transparent |
| | | bPEG | | | |
| 22 | POPC | DOPG/ | 1:20:2:2 | 29.2 nm | definite phase |
| | | 10k bPEG | | | separation; |
| | | | | | translucent |
| 23 | Poloxamine | none | 2:1 | 24.9 nm | transparent |
| 24 | Poloxamine | DOPG | 2:8:1 | 89.7 nm, 297.1 nm | transparent |
| 25 | Poloxamine | DOPG | 2:20:1 | 191.6 nm, 720.9 nm | slight settling |
| 26 | Poloxamine | DOPG | 2:40:1 | 170.7 nm, 434.7 nm | slight settling |
| 27 | POPC | Poloxamine | 1:20:4 | 25.3 nm | definite phase |
| | | | | | separation; |
| | | | | | translucent |
| 28 | POPC | Poloxamine | 1:20:2 | 30.0 nm | translucent |
| 29 | POPC | Poloxamine | 1:20:1 | 25.7 nm | definite phase |
| | | | | | separation; |
| | | | | | translucent |
| 30 | 10k bPEG | DOPG/ | 2:50:8:1 | 119.6 nm, 373.9 nm | slight settling |
| | | Poloxamine | | | |

*w/w ratio, wherein the first component is SN-38, and the subsequent components are the lipid listed in the component #1 and #2, respectively.
In cases of settling or phase separation, the phases are easily resuspended.
Poloxamine is dialyzed before formulation. See Examples for details.
10k bPEG refers to 10,000 Dalton branched polyethylene glycol.

TABLE 3

| | | | COMPOSITION | SIZE OF | |
|---|---|---|---|---|---|
| ID# | COMPONENT 1 | COMPONENT 2 | RATIO* | MAJOR PEAKS | 24-HR STABILITY |
| 31 | POPC | POPE- | 1:12:8 | 25.3 nm | slight settling |
| | | PEG5000 | | | |
| 32 | POPC | POPE- | 1:16:4 | 32.4 nm | slight settling |
| | | PEG5000 | | | |
| 33 | POPC | POPE- | 1:18:2 | 30.9 nm | slight settling |
| | | PEG5000 | | | |
| 34 | POPC | POPE- | 1:19:1 | 28.7 nm | slight settling; phases |
| | | PEG5000 | | | separate no |
| | | | | | resuspension |
| 35 | POPC | POPE- | 2:39:1 | 33.8 nm | slight settling; phases |
| | | PEG5000 | | | separate no |
| | | | | | resuspension |
| 36 | MRX-115* | none | 1:15 | 30.7 nm, 85.2 nm | slight settling |
| 37 | MRX-115 | none | 1:30 | 58.9 nm | slight settling |
| 38 | MRX-115 | none | 1:5 | 20.3 nm, 73.3 nm | slight settling |
| 39 | MRX-115u | none | 1:5 | 100.6 nm, 389.2 nm | slight settling |
| 40 | MRX-115u | none | 1:10 | 54.8 nm | slight settling |
| 41 | MRX-115u | none | 1:20 | 33.2 nm | slight settling; phases |
| | | | | | separate |

MRX-115 is a mixture of lipids comprised of DPPC:DPPA:DPPE-PEG5000 in the molar ratio of 80:10:15.
MRX-115u designates a mixture of DOPC:DOPA:DOPE-PEG5000, the corresponding unsaturated lipids in the same molar ratio.
Except where noted otherwise, all separated phases are readily resuspended.

Incorporation of an Acoustically Active Gas

In a further embodiment of the invention, the present formulations are made with small quantities of an acoustically active gas instilled therein. In order to instill the selected gas into the present formulations, a headspace of gas (preferably an insoluble gas) is applied atop the lyophilized composition in a closed container, which is then exposed to mild agitation during rehydration. Small quantities of gas will become entrapped in the interstices of the dispersion. The presence of the acoustically active gas is useful in conjunction with ultrasound imaging, as the gas-instilled dispersion produces an echogenic contrast that allows the drug to be tracked in the body. In addition, if a sufficient quantity of gas is entrapped in the formulation, therapeutic ultrasound can allow the microstructure to unfold at the locus where the ultrasound is applied, releasing the camptothecin analog and thus enhancing targeting effectiveness. The acoustically active gas lowers the cavitation threshold, i.e., the energy required for cavitation with ultrasound. Preferably, the cavitation energy used will be under about 1.5 MPa, and more preferably under about 1.0 MPa. The gas also effects dB reflectivity, and a gas concentration of about 1 mg per mL of particles will generally have a reflectivity approximately 2 dB higher than that of pure water.

In general, the amount of acoustically active gas that is imbibed by the particles of the formulation is approximately equal to the void space within the particles, which can be approximated by their density. For example, particles having a density of 0.10 will imbibe about 90 vol. % gas. Lower density particles will imbibe a higher volume of gas (e.g., 95 vol. % for particles having a density of 0.05), while higher density particles will imbibe a lower volume of gas (e.g., 85 vol. % for particles having a density of 0. 15). Gas may also adhere to the surface of the particles, typically up to about two times the volume of the particles. Normally, the amount of acoustically active gas that is employed is such that the gas-instilled formulation will contain at least about 5 vol. % gas, preferably about 10-15 vol. % gas.

Typical acoustically active gases are chemically inert gases having 1 to 12 carbon atoms, and particularly preferred acoustically active gases are perfluorocarbons, including saturated perfluorocarbons, unsaturated perfluorocarbons, and cyclic perfluorocarbons. The saturated perfluorocarbons, which are usually preferred, have the formula CₙF₂ₙ₊₂, where n is from 1 to 12, preferably 2 to 10, more preferably 4 to 8, and most preferably 5. Examples of suitable saturated perfluorocarbons are the following: tetrafluoromethane; hexafluoroethane; octafluoropropane; decafluorobutane; dodecafluoropentane; perfluorohexane; and perfluoroheptane. Saturated cyclic perfluorocarbons, which have the formula CₙF₂ₙ, where n is from 3 to 8, preferably 3 to 6, may also be preferred, and include, e.g., hexafluorocyclopropane, octafluorocyclobutane, and decafluorocyclopentane. Other gases that can be used include air, nitrogen, helium, argon, xenon, and other such gases.

Alternatively, a gaseous precursor can be used that is in the liquid state at room temperature and that is either (1) volatilized prior to introduction into the headspace above the lipid- and drug-containing dispersion, or (2) volatilized and instilled into a microemulsion that is then introduced into the lipid- and drug-containing dispersion. Suitable gaseous precursors are described, for example, in U.S. Pat. No. 5,922,304 to Unger, and include, without limitation: hexafluoro acetone, isopropyl acetylene, allene, tetrafluoroallene, boron trifluoride, isobutane, 1,2-butadiene, 2,3-butadiene, 1,3-butadiene, 1,2,3-trichloro-2-fluoro-1,3-butadiene, 2-methyl-1,3-butadiene, hexafluoro-1,3-butadiene, butadiyne, 1-fluoro-butane, 2-methyl-butane, decafluorobutane, 1-butene, 2-butene, 2-methyl-1-butene, 3-methyl-1-butene, perfluoro-1-butene, perfluoro-2-butene, 4-phenyl-3-butene-2-one, 2-methyl-1-butene-3-yne, butyl nitrate, 1-butyne, 2-butyne, 2-chloro-1,1,1,4,4,4-hexafluorobutyne, 3-methyl-1-butyne, perfluoro-2-butyne, 2-bromobutyraldehyde, carbonyl sulfide, crotononitrile, cyclobutane, methyl-cyclobutane, octafluorocyclobutane, perfluorocyclobutene, 3-chlorocyclopentene, octafluorocyclopentenecyclopropane, 1,2-dimethyl-cyclopropane, 1,1-dimethylcyclopropane, 1,2-dimethylcyclopropane, ethylcyclopropane, methylcyclopropane, diacetylene, 3-ethyl-3-methyl diaziridine, 1,1,1-trifluorodiazoethane, dimethyl amine, hexafluorodimethylamine, dimethylethylamine, bis(dimethylphosphine)amine, perfluorohexane, 2,3-dimethyl-2-norbornane, perfluorodimethylamine, dimethyloxonium chloride, 1,3-dioxolane-2-one, 4-methyl-1,1,1,2-tetrafluoroethane, 1,1,1-trifluoroethane, 1,1,2,2-tetrafluoroethane, 1,1,2-trichloro-1,2,2-trifluoro-ethane, 1,1-dichloroethane, 1,1-dichloro-1,2,2,2-tetrafluoroethane, 1,2-difluoroethane, 1-chloro-1,1,2,2,2-pentafluoroethane, 2-chloro-1,1-difluoroethane, 1,1-dichloro-2-fluoroethane, 1-chloro-1,1,2,2-tetrafluoroethane, 2-chloro-1,1-difluoroethane, chloroethane, chloropenta-fluoroethane, dichlorotrifluoroethane, fluoroethane, hexafluoroethane, nitropentafluoroethane, nitrosopentafluoroethane, perfluoroethylamine, ethyl vinyl ether, 1,1-dichloroethane, 1,1-dichloro-1,2-difluoroethane, 1,2-difluoroethane, methane, trifluoromethanesulfonylchloride, trifluoromethane-sulfonylfluoride, bromodifluoronitrosomethane, bromofluoromethane, bromochloro-fluoromethane, bromotrifluoromethane, chlorodifluoronitromethane, chlorodinitromethane, chlorofluoromethane, chlorotrifluoromethane, chlorodifluoromethane, dibromodifluoromethane, dichlorodifluoromethane, dichlorofluoromethane, difluoromethane, difluoroiodo-methane, disilanomethane, fluoromethane, iodomethane, iodotrifluoromethane, nitrotrifluoromethane, nitrosotrifluoromethane, tetrafluoromethane, trichlorofluoromethane, trifluoromethane, 2-methylbutane, methyl ether, methyl isopropyl ether, methyllactate, methylnitrite, methylsulfide, methyl vinyl ether, neon, neopentane, nitrogen (N₂), nitrous oxide, 1,2,3-nonadecane-tricarboxylic acid-2-hydroxytrimethylester, 1-nonene-3-yne, oxygen (O₂), 1,4-pentadiene, n-pentane, perfluoropentane, 4-amino-4-methylpentan-2-one, 1-pentene, 2-pentene (cis), 2-pentene (trans), 3-bromopent-1-ene, perfluoropent-1-ene, tetrachlorophthalic acid, 2,3,6-trimethylpiperidine, propane, 1,1,1,2,2,3-hexafluoropropane, 1,2-epoxypropane, 2,2-difluoropropane, 2-aminopropane, 2-chloropropane, heptafluoro-1-nitropropane, heptafluoro-1-nitrosopropane, perfluoropropane, propene, hexafluoropropane, 1,1,1,2,3,3-hexa-fluoro-2,3 dichloropropane, 1-chloropropane, chloropropane (trans), 2-chloropropane, 3-fluoropropane, propyne, 3,3,3-trifluoropropyne, 3-fluorostyrene, sulfur hexafluoride, sulfur (di)-decafluoride(S₂F₁₀), 2,4-diaminotoluene, trifluoroacetonitrile, trifluoromethyl peroxide, trifluoromethyl sulfide, tungsten hexafluoride, vinyl acetylene, vinyl ether, and xenon.

Utility:

The formulations of the invention are used to treat a mammalian individual, generally a human patient, suffering from a condition, disease, or disorder that is responsive to systemic administration of a camptothecin derivative. The formulations may be administered orally, parenterally, topically, transdermally, rectally, vaginally, by inhalation, intraocularly, in an implanted reservoir (i.e., in a sustained release depot for subcutaneous or intramuscular administration), or as a packing material for wounds and fractures. The term “parenteral” as used herein is intended to include subcutaneous, intravenous, intramuscular, intra-arterial, intrathecal, and intraperitoneal injection, and the formulation may be injected as either a bolus or an infusion.

Table 4 shows comparative data from production of nanoparticles of camptothecin and SN-38. Several surprising and unexpected findings are evident. Firstly, nanoparticles prepared with camptothecin are much larger than nanoparticles prepared with SN-38. Secondly, nanoparticles prepared with the addition of an anionic lipid (phosphatidylglycerol) are much smaller than the particles prepared without the anionic lipid. A neutral lipid, by comparison, prepares much larger particles, which settle quickly. The particles prepared with phosphatidylglycerol remain in solution as a homogeneous suspension.

TABLE 4

| | | | | 24 HOUR |
|---|---|---|---|---|
| ID# | COMPONENTS | RATIO | POST FLUIDIZATION | STABILITY |
| 42 | POPG: | SN-38:POPG: | Intensity Weighted: | Trace amount white |
| | 123.8 mg | Poloxamer | 105.0 nm 30.7% | and brownish |
| | Poloxamer 188: | 2:8:1 | 286.4 nm 69.3% | sediment after 24 hrs. |
| | 15.0 mg | | Volume Weighted: | No phase separation, |
| | SN-38: | | 102.2 nm 67.1% | stable. |
| | 30 mg | | 289.2 nm 32.9% | |
| | 60 ml 0.50 mg/ml Stock | | Number Weighted: | |
| | Solution | | 98.8 nm 98.0% | |
| | | | 285.7 nm 2.0% | |
| 43 | POPC: | SN-38:POPC: | Intensity Weighted: | Suspension settles |
| | 124.2 mg | Poloxamer | 268.5 nm 26.9% | loosely to bottom of |
| | Poloxamer 188: | 2:8:1 | 942.3 nm 73.1% | vial leaving a clear |
| | 15.0 mg | | Volume Weighted: | supernatant after 24 |
| | SN-38: | | 272.5 nm 6.7% | hrs. Re-suspends |
| | 30 mg | | 962.6 nm 93.3% | easily. |
| | 60 ml 0.50 mg/ml Stock | | Number Weighted: | |
| | Solution | | 266.9 nm 76.4% | |
| | | | 952.6 nm 23.6% | |
| 44 | Poloxamer 188: | SN-38:Poloxamer | Intensity Weighted: | Trace amount white |
| | 15.0 mg | 2:1 | 234.8 nm 99.6% | sediment after 24 hrs. |
| | SN-38: | | Volume Weighted: | No phase separation, |
| | 30 mg | | 235.7 nm 95.0% | stable. |
| | 60 ml 0.50 mg/ml Stock | | 1359.9 nm 5.0% | |
| | Solution | | Number Weighted: | |
| | | | 197.5 nm 100.0% | |
| 45 | POPG: | CPT:POPG: | Intensity Weighted: | No sedimentation, |
| | 121.2 mg | Poloxamer | 254.8 nm 82.9% | stable for less than 24 |
| | Poloxamer 188: | 2:8:1 | 1859.1 nm 17.1% | hrs. |
| | 15.0 mg | | Volume Weighted: | |
| | Camptothecin: | | 257.6 nm 35.0% | |
| | 30 mg | | 1783.1 nm 65.0% | |
| | 50 ml 0.63 mg/ml Stock | | Number Weighted: | |
| | Solution | | 251.5 nm 99.4% | |
| 46 | POPC: | CPT:POPC: | Intensity Weighted: | Suspension settles |
| | 121.8 mg | Poloxamer | 256.5 nm 28.0% | loosely to bottom of |
| | Poloxamer 188: | 2:8:1 | 989.8 nm 72.0% | vial leaving a |
| | 15.0 mg | | Volume Weighted: | supernatant that is |
| | Camptothecin: | | 259.5 nm 9.6% | more transparent than |
| | 30 mg | | 1002.9 nm 90.4% | the original |
| | 50 ml 0.63 mg/ml Stock | | Number Weighted: | suspension, less than |
| | Solution | | 254.4 nm 86.0% | 24 hrs. Re-suspends |
| | | | 984.9 nm 14.0% | easily. |
| 47 | Poloxamer 188: | CPT:Poloxamer | Intensity Weighted: | No sedimentation, |
| | 15.0 mg | 2:1 | 309.9 nm 100.0% | stable for less than 24 |
| | Camptothecin: | | Volume Weighted: | hrs. |
| | 30 mg | | 400.0 nm 100.0% | |
| | 50 ml 0.63 mg/ml Stock | | Number Weighted: | |
| | Solution | | 233.8 nm 100.0% | |

The levels of anticancer efficacy for the formulations of the invention represent a significantly improved profile when compared with CAMPTOSAR®, a water-soluble, FDA-approved form of camptothecin. Details of the efficacy studies and an interpretation of the results are presented in Examples 11 and 12.

The present formulations are also useful as packing materials for wounds and fractures, and as coating materials for endoprostheses such as stents, grafts, and joint prostheses. It is known that restenosis (narrowing of the blood vessels) may occur after angioplasty, placement of a stent, and/or other coronary intervention procedures, as a result of fibroblast proliferation and smooth muscle hypertrophy. Thus, the formulations of the invention may be used as coating materials for endoprostheses to provide local drug delivery following coronary intervention to, for example, prevent or inhibit restenosis.

It is to be understood that while the invention has been described in conjunction with the preferred specific embodiments thereof, the foregoing description as well as the examples that follow are intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entirety.

Experimental

The following examples are put forth so as to provide those of ordinary skill in the art with a complete disclosure and description of how to prepare and use the formulations disclosed and claimed herein. Efforts have been made to ensure accuracy with respect to numbers (e.g., amounts, temperatures, etc.) but some errors and deviations should be accounted for. Unless indicated otherwise, parts are parts by weight, temperature is in degrees Celsius (° C.), and pressure is at or near atmospheric.

Also, in these examples and throughout this specification, the abbreviations employed have their generally accepted meanings, as follows:

g=gram

mL=milliliter

mmol=millimole

nm=nanometer

μL=microliter

μm=micrometer

### EXAMPLE 1

### PREPARATION OF FORMULATION 24 IN TABLE 2

Procedure for Making Poloxamine-Stabilized Composition:

Twenty-five milliliters of SN-38 formulation were made (composition ratio 2:8:1 SN-38:DOPG:poloxamine (TETRONIC® 908), 1 mg/mL SN-38, 4 mg/mL DOPG, 0.5 mg/mL poloxamine) using a small microfluidizer.

120 mg of DOPG was dissolved with 40 mL of t-butanol in a 250 mL round-bottom flask by heating for a few minutes on a Rotovap. SN-38 stock solution, 0.5 mg/mL in dichloromethane, was added to the DOPG solution in the flask until the desired SN-38 concentration was reached. (60 mL of stock solution was used to achieve a 1.0 mg/mL SN-38 concentration.) The flask was placed on a Rotovap, and heated for 15 min to remove the dichloromethane. The flask was flash-frozen with liquid nitrogen and freeze-dried overnight.

The formulation was rehydrated with 25 mL of un-buffered poloxamine solution (0.5 g poloxamine, diluted to 1 L with purified water) and allowed to sit for 30-60 min, shaking occasionally, until no large clumps of material were present. A microfluidizer was rinsed with the rehydration solution to fill 5 mL of microfluidizer dead volume and to achieve 30 mL final formulation rehydration volume. The solution was microfluidized for 20 min at a pressure of approximately 50 psig. The resulting suspension was faintly yellow and translucent with some birefringence. Some settling of particulate matter occurred after 72 hrs refrigeration.

### EXAMPLE 2

### PREPARATION OF FORMULATION 33 IN TABLE 3

Procedure for Making 25 mL of SN-38 Formulation(Composition Ratio 1:18:2 SN-38:POPC:DOPG, 1mg/mL SN-38, 18mg/mL POPC, 2 mg/mL DOPG):

540.0 mg of POPC and 60.0 mg DOPG were dissolved in 40 mL of t-butanol in a 250 mL round-bottom flask by heating for a few minutes on a Rotovap. Note that heating was only used to speed dissolution. To the POPC/DOPG solution was added 0.5 mg/mL SN-38 stock solution until the desired SN-38 concentration was reached. (60 mL of stock solution was used to achieve a 1.0 mg/mL SN-38 concentration.) The flask was placed on a Rotovap, and heated for 15 min to remove the dichloromethane. The flask was the flash-frozen with liquid nitrogen and freeze-dried overnight.

The freeze-dried formulation was then rehydrated with a 4.1 mL 0.1 M citric acid solution. The citric acid solution was made using 5.9 mL 0.1 M sodium citrate, diluted to 1 L with purified water. The pH was adjusted to 5±0.1. The hydrated formulation was allowed to sit for 20-30 min, shaking occasionally. The formulation was then sonicated for 10-20 min until no large clumps of material were present. A microfluidizer was rinsed with rehydration solution to fill 5 mL of microfluidizer dead volume and achieve 30 mL final formulation rehydration volume. The solution was then microfluidized for 20 min at a pressure of approximately 50 psig. 25 mL of formulation was collected from the microfluidizer via syringe. The resulting suspension was pale-yellow, translucent, non-birefringent, and stable under prolonged refrigeration.

### EXAMPLE 3

### PREPARATION OF FORMULATION 41 IN TABLE 3

Unsaturated Stabilized Lipid Blend Formulation:

Procedure for making 25 mL of SN-38 (formulation #41 from Table 3) (composition ratio 1:20 SN-38: unsaturated blend, 1 mg/mL SN-38, 20 mg/mL mixture of lipid blend containing POPC:POPA:POPE-PEG5000 in the molar ratio of 80:10:15.) using a small microfluidizer.

600.0 mg of an unsaturated blend (blend comprised of 324.0 mg POPC, 240 mg POPE-PEG5000, and 36 mg POPA) was dissolved in 40 mL of t-butanol in a 250 mL round-bottom flask by heating for a few minutes on a Rotovap. SN-38 stock solution, 0.5 mg/mL, was added to the unsaturated lipid blend solution in the flask until the desired SN-38 concentration was reached. (60 mL of stock solution was used to achieve a 1.0 mg/mL SN-38 concentration.) The flask was placed on a Rotovap, and heated for 15 min to remove the dichloromethane. The flask was then flash-frozen with liquid nitrogen and freeze-dried overnight.

The formulation was then rehydrated with 25 mL of a 4.1 mL 0.1 M citric acid solution (5.9 mL 0.1 M sodium citrate, diluted to 1 L with purified water) and the pH adjusted to 5±0.1. The hydrated formulation was allowed to sit for 20-30 min, with occasional shaking. The formulation was then sonicated for 20-30 min until no large clumps of material were present. A microfluidizer was then rinsed with rehydration solution to fill 5 mL of microfluidizer dead volume and achieve 30 mL final formulation rehydration volume. The solution was then microfluidized for 20 min at a pressure of approximately 50 psi. 25 mL of the formulation was collected from the microfluidizer via syringe.

The resulting suspension was pale-yellow, transparent, non-birefringent, and settled only slightly after 24 hrs under prolonged refrigeration.

### EXAMPLE 4

### PREPARATION OF FORMULATION 42 IN TABLE 4

The procedure described in Example 1 was duplicated, substituting an equivalent amount of Pluronic-F68 (Poloxamer 188) for poloxamine.

### EXAMPLE 5

### PREPARATION OF FORMULATION 43 IN TABLE 4

The procedure was identical to that in Example 4, but substituted POPC for POPG.

### EXAMPLE 6

### PREPARATION OF FORMULATION 44 IN TABLE 4

The procedure from Example 1 was followed for the initial solubilization of SN-38 to the same concentration. The same flash-freezing and freeze-drying procedures were followed. In this instance, however, the freeze-dried formulation was rehydrated with 25 mL of an un-buffered poloxamine solution (0.5 g poloxamine, diluted to 1 L with purified water).

The hydrated formulation was allowed to sit for 30-60 min, shaking occasionally, until no large clumps of material were present. A microfluidizer was rinsed with rehydration solution to fill 5 mL of microfluidizer dead volume and achieve 30 mL final formulation rehydration volume and the solution was microfluidized for 20 min at a pressure of approximately 50 psi. 25 mL of the formulation was collected from the microfluidizer via syringe.

### EXAMPLE 7

### PREPARATION OF FORMULATION 45 IN TABLE 4

The procedure was identical to that followed in Example 4 except for the substitution of camptothecin for SN-38.

### EXAMPLE 8

### PREPARATION OF FORMULATION 46 IN TABLE 4

The procedure was identical to that followed in Example 5 except for the substitution of camptothecin for SN-38.

### EXAMPLE 9

### PREPARATION OF FORMULATION 47 IN TABLE 4

The procedure was identical to that followed in Example 6 except for the substitution of camptothecin for SN-38.

### EXAMPLE 10

### LYOPHILIZATION PROCEDURE FOR FORMULATION 23

A formulation containing SN-38 (Abatra Technology Co. LTD, Xi'an, China), poloxamine 908 (BASF Corp., Mount Olive, N.J.), and sucrose (Spectrum Laboratory Products, Gardena, Calif.) was lyophilized in the following manner:

The SN-38/poloxamine formulation was prepared using the standard method of lyophilization from t-butanol/dichloromethane, and rehydrated in purified water. The rehydrated formulation was microfluidized, and sucrose was added after the fluidization step. 1 mL aliquots of the formulation were transferred to 2 cc, 13 mm flint glass tubing vials (Helvoet, Pennsauken, N.J.). The vials were stoppered with 13 mm lyo-type rubber stoppers (Daikyo-Seiko, Japan) in the lyo-position and placed in a Unitop SQ Drying Stoppering chamber equipped with a Freezemobile research-scale freeze-dryer (Virtis Company, Gardiner, N.Y.). The formulation was then lyophilized using a standard 2-step lyophilization cycle. The resulting product was a uniform yellowish cake that rehydrated readily with gentle shaking.

For formulations containing both phospholipids and poloxamine or poloxamer, the lipids and camptothecin drugs were coformulated prior to lyophilization and the poloxamine/poloxamer was added during rehydration.

### EXAMPLE 11

### ALTERNATIVE FORMULATION METHOD

A formulation containing SN-38 (Abatra Technology Co. LTD, Xi'an, China), polaxamine 908 (BASF Corp., Mount Olive, N.J.), and sucrose (Spectrum Laboratory Products, Gardena, Calif.) was processed in the following manner:

The lyophilization steps from Example 10 were followed, except the lyophilisate was resuspended in 0.01 M citrate, pH 5. Half of the rehydrated formulation was then microfluidized for 20 min. The other aliquot was extruded using an SP extruder (SP Pharmaceuticals, Albuquerque, N.Mex.) fitted with a series of 200 nm, 80 nm, 50 nm and 90 nm polycarbonate filters (Whatman, Kent, UK). The fluidized sample was then extruded the same way and particle sizes were compared. Both formulations showed a volume-weighted size of less than 300 nm.

### EXAMPLE 12

### ANTI-TUMOR EFFICACY OF SN-38 FORMULATIONS

A culture of HT-29 human colon adenocarcinoma cells from ATCC was grown in McCoy's 5a medium with L-glutamine, sodium bicarbonate, and 10% fetal calf serum at 37° C. under an atmosphere of 5% CO₂. Cells were collected with trypsin-EDTA and spun at 250× g. A final dilution was prepared at 5 million cells per liter.

Two 50 microliter injections of cells were given to nude mice to form tumors in the upper leg region. At seven days following inoculation, the mice had treatments initiated with 500 microliter inocula of each formulation. Control mice were untreated. For CAMPTOSAR®, the concentrations of active compound were adjusted to be approximately 6× higher than for the amounts of SN-38 in experimental animals. Also, CAMPTOSAR® was administered daily while the SN-38 treated animals were dosed twice weekly. After 21 days of treatment, no further inocula of CAMPTOSAR® or SN-38 formulations were administered in order to assess the duration of efficacy. The experiments were terminated when tumor growth reached 1 gram.

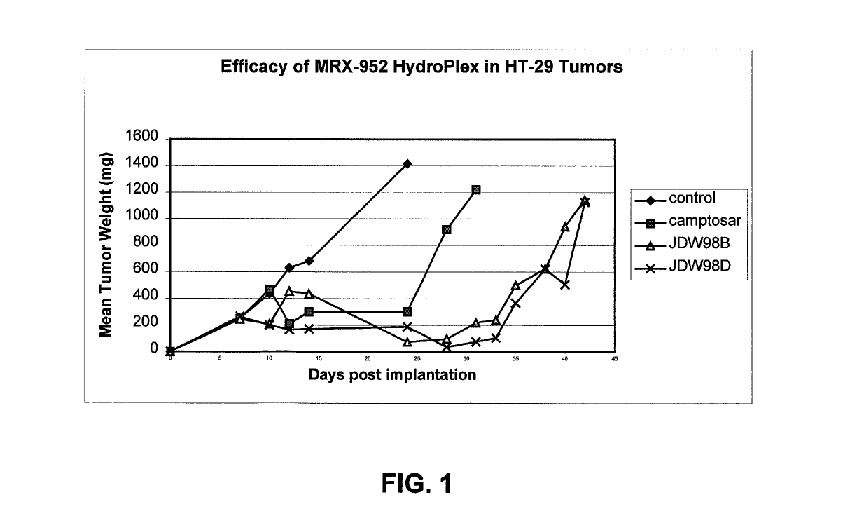

Results of the study comparing untreated (control), CAMPTOSAR®-treated and SN-38 formulations are shown in FIG. 1. JDW98B and JDW98D are formulations of SN-38 with branched polyethylene glycol, wherein 98B is comprised of SN38:10 kD bPEG:DOPG at a ratio of 1:50:4 and JDW98D is comprised of SN38:10 kD bPEG:DOPG at a ratio of 1:25:2. It is evident that after an initial lag time of 1-2 weeks SN-38-containing formulations are more effective at inhibiting the growth of tumor masses than CAMPTOSAR®. Also notable is the duration of the efficacy. Tumors in mice treated with CAMPTOSAR® begin to increase in weight within 24 hrs after the last inoculation. The resumption of tumor growth in animals treated with SN-38:branched PEG formulations does not begin for 10 days after the last dose, indicating a sustained release profile in circulation. Toxicity data (not shown) as measured by total weight gain/loss of animals during the treatment intervals shows that SN-38:branched PEG is well tolerated. Animals returned to full initial weights after cessation of treatment.

### EXAMPLE 13

### COMPARATIVE EFFICACIES OF VARIOUS LIPIDIC STABILIZED SN-38 FORMULATIONS

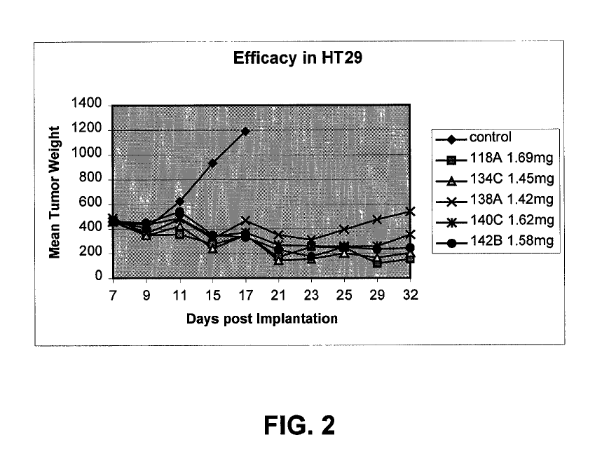

Another set of experiments was conducted, run identically to those described in Example 12 but with all test formulations containing SN-38. Formulation 118A corresponds to #23 from Table 2 above, containing no lipidic stabilizers. Formulation 138A corresponds to #36 from Table 3; formulation 140° C. corresponds to #2 from Table 1, and formulation 142B corresponds to #39 from Table 3. All animals were tested in groups of four mice (eight tumors). It is apparent from FIG. 2 that all lipidic and non-lipidic stabilized formulations of SN-38 exhibit comparable and statistically indistinguishable antitumor efficacy, which in all cases is positive.

### EXAMPLE 14

### COMPARATIVE SERUM STABILITY OF CAMPTOTHECIN AND SN-38

A quasi-serum is made by dissolving HSA (human serum albumin) in water to a concentration of 20 mg/mL. The solution is heated to 37° C., and aliquots of 50 microliters of camptothecin or SN-38 formulations are added to produce a final dilution of 1:40. Kinetics of lactone ring opening are monitored by HPLC. Testing over 50 samples containing unstabilized camptothecin and a like number of stabilized SN-38, it was found that the average equilibrium percentage of lactone ring form was approximately 10% for camptothecin and over 60% for SN-38. The range for camptothecin samples was from 3-26% and for SN-38 from 50-94%. Clearly, the lactone ring is more stable to opening and subsequently less susceptible to loss of bioactivity in all SN-38 formulations compared to camptothecin formulations. Thus, it can be concluded that the chemical stability is greater and the therapeutic window for SN-38 is significantly enhanced in contrast to camptothecin.

### EXAMPLE 15

### FORMULATION OF SN-38 BY SUPERCRITICAL FLUID TECHNOLOGY

Each of the formulations enumerated in Tables 1-4 can be produced by alternative methodologies to those described in the Examples above. One preferred formulation methodology utilizes supercritical fluid solubilization followed by extrusion through a nozzle. In this method, the lipidic suspension of SN-38 and lipids as described in Example 1 is stirred into liquid carbon dioxide. Following this procedure, the material is collected and microfluidized, with subsequent procedures used as described above. As one skilled in the art would recognize supercritical fluid processing may also be performed with other solvents and cosolvents besides carbon dioxide, depending upon the solubility properties of the drug and the stabilizing materials.

### EXAMPLE 16

### FORMULATION OF 7-ETHYLCAMPTOTHECIN NANOPARTICLES

An unsaturated lipid blend formulation is made using 7-ethylcamptothecin (Boehringher-Ingelheim, Ingelheim, Germany) by substituting 7-ethylcamptothecin for SN-38 and using the method described in Example 1.

### EXAMPLE 17

### ELECTRON MICROSCOPY OF SN-38 FORMULATIONS

Cryo-electron microscopy was performed on frozen hydrated specimens. In this example, formulation #37 from table 3 was used. A thin layer (200-400 nm) of a suspension of the nanoparticles was created by applying 4 ul (microliter) droplets of the suspension to the coated surface of “lacey” carbon-coated EM grids and blotting away the excess liquid with a piece of filter paper from the back side of the grids. Plunging the grids into liquid propane cooled to near liquid nitrogen temperature vitrified the thin layer of suspended nanoparticles. The specimens were stored in special grid holders in liquid nitrogen until cryotransfer and observation at −170C on a GATAN model 626 TEM cold stage, in a Philips 420 TEM.

We claim:

1. A pharmaceutical formulation comprising:
a camptothecin analog;
a stabilizing agent that stabilizes the camptothecin analog but does not covalently bind thereto;
an optional targeting ligand; and
an optional excipient.

2. The formulation of claim 1, wherein the stabilizing agent comprises a polymer, and/or a lipid.

3. The formulation of claim 2, wherein the stabilizing agent comprises a polymer.

4. The formulation of claim 3, wherein the polymer is selected from the group consisting of polyethylene glycol, polyglycolide, polyvinyl alcohol, polyvinyl pyrrolidone, polylactide, poly(lactide-co-glycolide), polysorbate, polyethylene oxide, polypropylene oxide, poly(ethylene oxide-co-propylene oxide), poloxamer, poloxamine, poly(oxyethylated) glycerol, poly(oxyethylated) sorbitol, poly(oxyethylated) glucose, and derivatives, mixtures, and copolymers thereof.

5. The formulation of claim 3, wherein the polymer comprises a branched polymer.

6. The formulation of claim 4, wherein the polymer comprises poloxamer.

7. The formulation of claim 4, wherein the polymer comprises poloxamine.

8. The formulation of claim 4, wherein the polymer comprises polyethylene glycol or polypropylene glycol.

9. The formulation of claim 8, wherein the polymer is selected from branched polyethylene glycol, star polyethylene glycol, linear polyethylene glycol, and combinations thereof, and is optionally covalently bound to at least one phospholipid moiety.

10. The formulation of claim 8, wherein the polyethylene glycol is functionalized to contain at least one sulfhydryl, amino, lower alkoxy, carboxylate, or phosphonate moiety.

11. The formulation of claim 8, wherein the polyethylene glycol or polypropylene glycol contains a hydrolyzable linkage.

12. The formulation of claim 8, wherein the polyethylene glycol is bonded to a phospholipid moiety.

13. The formulation of claim 12, wherein the polyethylene glycol ranges in size from approximately 350 Daltons to approximately 7000 Daltons.

14. The formulation of claim 13, wherein the polyethylene glycol ranges in size from approximately 750 Daltons to approximately 5000 Daltons.

15. The formulation of claim 2, wherein the stabilizing agent comprises a lipid.

16. The formulation of claim 15, wherein the lipid is comprised of a member of the group consisting of natural phospholipids, chemically and enzymatically modified phospholipids, and synthetic phospholipids.

17. The formulation of claim 16, wherein the lipid comprises a natural phospholipid.

18. The formulation of claim 16, wherein the lipid comprises a synthetic phospholipid.

19. The formulation of claim 16, wherein the lipid is selected from the group consisting of diacyl phosphatidylcholines, diacyl phosphatidylethanolamines, diacyl phosphatidylserines, diacyl phosphatidylinositols, diacyl phosphatidic acids, phosphorylated diacylglycerides, and combinations thereof.

20. The formulation of claim 19, wherein the lipid is a phosphorylated diacylglyceride.

21. The formulation of claim 20, wherein the stabilizing agent is selected from the group consisting of palmitoyloleylphosphatidylglycerol, dipalmitoyl phosphatidylethanolamine, 1-palmitoyl-2-oleoylphosphatidyl-ethanolamine, and combinations thereof.

22. The formulation of claim 19, wherein the stabilizing agent is a diacyl phosphatidylcholine.

23. The formulation of claim 22, wherein the stabilizing agent is selected from the group consisting of palmitoyl-oleoyl phosphatidylcholine, dioleoyl phosphatidylcholine, dilauroyl phosphatidylcholine, dimyristoyl phosphatidylcholine, dipalmitoyl phosphatidylcholine, distearoyl phosphatidylcholine, and mixtures thereof.

24. The formulation of claim 1, wherein the stabilizing agent comprises a polymer and lipid.

25. The formulation of claim 1, wherein the optional excipient is present.

26. The formulation of claim 25, wherein the excipient is selected from the group consisting of polyhydroxyalcohols, saccharides, liquid polyethylene glycols, propylene glycol, glycerol, ethyl alcohol, and combinations thereof.

27. The formulation of claim 1, wherein the camptothecin analog has the structure of formula (I) 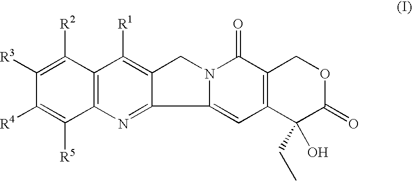
wherein R¹, R², R³, R⁴, and R⁵ are independently selected from the group consisting of H, C₁₋₆ alkyl, C₁₋₆ alkoxy, acyloxy, hydroxyl, sulfhydryl, acyl, halo, amido, C₁₋₆ alkylamido, amino, nitro, and cyano, or R¹ and R² and/or R³ and R⁴ may together form a substituted or unsubstituted five- or six-membered cyclic group containing up to 2 heteroatoms selected from the group consisting of O, S, and N.

28. The formulation of claim 27, wherein R¹, R², R³, R⁴, and R⁵ are independently selected from the group consisting of H, C₁₋₆ alkyl, C₁₋₆ alkoxy, acyloxy, hydroxyl, sulfhydryl, acyl, halo, amido, C₁₋₆ alkylamido, amino, nitro, and cyano.

29. The formulation of claim 28, wherein R¹ is C₁₋₆ alkyl, and R², R³, R⁴, and R⁵ are independently selected from the group consisting of H, C₁₋₆ alkyl, C₁₋₆ alkoxy, acyloxy, hydroxyl, sulfhydryl, acyl, halo, amido, C₁₋₆ alkylamido, amino, nitro, and cyano.

30. The formulation of claim 29, wherein R³ is hydroxyl, and R², R⁴, and R⁵ are independently selected from the group consisting of H, C₁₋₆ alkyl C₁₋₆ alkoxy, acyloxy, hydroxyl, sulfhydryl, acyl, halo, amido, C₁₋₆ alkylamido, amino, nitro, and cyano.

31. The formulation of claim 27, wherein R³ is hydroxyl, and R¹, R², R⁴, and R⁵ are independently selected from the group consisting of H, C₁₋₆ alkyl C₁₋₆ alkoxy, acyloxy, hydroxyl, sulfhydryl, acyl, halo, amido, C₁₋₆ alkylamido, amino, nitro, and cyano.

32. The formulation of claim 37, wherein R², R⁴, and R₅ are H, such that the camptothecin analog has the structure of formula (II)

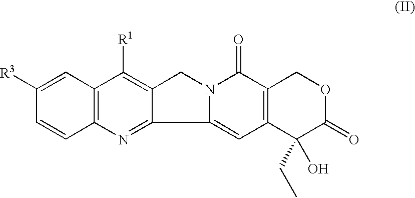

33. The formulation of claim 32, wherein R₁ is C₁₋₆ alkyl and R³ is hydroxyl, sulfhydryl, or amino.

34. The formulation of claim 33, wherein R³ is hydroxyl.

35. The formulation of claim 34, wherein the camptothecin analog is 7-ethyl-10-hydroxyl camptothecin.

36. The formulation of claim 1, wherein the formulation is in the form of an aqueous suspension and further comprises an aqueous vehicle.

37. The formulation of claim 36, wherein the aqueous vehicle is water, an isotonic diluent, or a buffer solution.

38. The formulation of claim 1, wherein the formulation is particulate.

39. The formulation of claim 38, wherein the formulation is comprised of particles that have an average size in the range of approximately 1 nm to approximately 1 μm.

40. The formulation of claim 39, wherein the average size of the particles is in the range of approximately 30 nm to approximately 250 nm.

41. The formulation of claim 36, wherein the aqueous suspension further comprises an acoustically active gas.

42. A method for making a nanoparticulate formulation of a camptothecin analog, comprising:
(a) admixing, in a solvent, a camptothecin analog and a stabilizing agent that stabilizes the camptothecin analog but does not covalently bond thereto;
(b) removing the solvent in a manner effective to provide a dry formulation of the camptothecin analog; and
(c) rehydrating the dry formulation to provide the nanoparticulate formulation.

43. The method of claim 42, wherein the solvent is removed by lyophilization.

44. The method of claim 42, wherein the solvent is removed by spray drying.

45. The method of claim 42, wherein (b) comprises removing the solvent by rotary evaporation, thereby providing an agglomerated intermediate product, and wherein the method further comprises (b′) deagglomerating the intermediate product using a procedure effective to provide the nanoparticulate formulation of the camptothecin analog.

46. The method of claim 42, wherein prior to (a), the step is added of dissolving the camptothecin analog in a first solvent to form a first solution and dissolving the stabilizing agent in a second solvent to form a second solution, and (a) comprises admixing the first solution with second solution.

47. The method of claim 42, wherein an additional component of the stabilizing agent is added during step (c).

48. The method of claim 47, wherein the additional component of the stabilizing agent is a poloxamer and/or a poloxamine.

49. A nanoparticulate formulation of a camptothecin analog prepared according to the method of claim 42.

50. A method for delivering a drug to a mammalian individual to achieve a desired therapeutic effect, comprising administering to the individual a therapeutically effective amount of the formulation of claim 1.

51. The method of claim 50, wherein administration is parenteral.

52. The method of claim 51, wherein administration is intravenous.

53. The method of claim 50, wherein administration is oral.

54. A method for treating an individual suffering from cancer, comprising administering to the individual a pharmaceutical formulation of: (a) drug-containing particles comprised of (i) a stabilizing agent, (ii) a camptothecin analog that is entrapped by but not covalently bound to the stabilizing agent, optionally (iii) a targeting ligand, and optionally (iv) an excipient selected from the group consisting of saccharides, liquid polyethylene glycols, propylene glycol, glycerol, ethyl alcohol, and combinations thereof, in (b) an aqueous vehicle suitable for parenteral drug administration.

55. The method of claim 54, wherein the formulation is administered parenterally and the vehicle is suitable for parenteral administration.
